(19) United States
(12) Patent Application Publication
(10) Pub. No.: US 2006/0182765 A1
(43) Pub. Date: Aug. 17, 2006

(54) Adjuvant activities of B pentamers of LT-IIa and LT-IIb enterotoxin

(76) Inventors: Terry D. Connell, Williamsville, NY (US); Michael W. Russell, East Amherst, NY (US); Hesham Nawar, Buffalo, NY (US); Georgios Hajishengallis, Louisville, KY (US)

Correspondence Address: HODGSON RUSS LLP, ONE M & T PLAZA, SUITE 2000, BUFFALO, NY, 14203-2391, US

(21) Appl. No.: 11/354,497

(22) Filed: Feb. 15, 2006

Related U.S. Application Data
(60) Provisional application No. 60/653,235, filed on Feb. 15, 2005.

(51) Int. Cl. A61K 39/02

(52) U.S. Cl. 424/235.1

(57) ABSTRACT

The present invention provides a method for enhancing an immunological response to an antigen. The method comprises administering to an individual a composition comprising an antigen and an isolated LT-IIa-B pentamer or a mutant thereof, or an isolated LT-IIb-B pentamer or a mutant thereof. The selected LT-II-B pentamer acts as an adjuvant to enhance the immunological response to the co-administered antigen.

This application claims priority to U.S. provisional patent application Ser. No. 60/653,235, filed Feb. 15, 2005, the disclosure of which is incorporated herein by reference.

This work was supported by Grant nos. DE13833, DE015254, DE06746 from the National Institutes of Health. The Government has certain rights in the invention.

### FIELD OF THE INVENTION

The present invention relates generally to the field of adjuvants and more particularly to adjuvant activities of B pentamers of LT-IIa and LT-IIb enterotoxin.

### DISCUSSION OF RELATED ART

Since mucosal surfaces represent the major entry route of many microbial pathogens, it is important that prospective vaccines stimulate appropriate immune response at these sites.

However, the mucosal immune system usually requires the aid of immune stimulating agents (i.e., adjuvants) to generate robust immunity and long-lived memory responses to an antigen. The type I heat-labile enterotoxins produced by Vibrio cholerae and Escherichia coli (CT and LT-I, respectively) have been extensively characterized as mucosal adjuvants in a variety of animals (Harandi, A. M., et al., 2003, Curr. Opin. Investig. Drugs 4:156-161). The immunomodulatory activities of a second class of heat-labile enterotoxins of E. coli have also been described. This second class consists of LT-IIa and LT-IIb, two heat-labile enterotoxins from E. coli which can be distinguished from LT-I by a variety of antigenic and genetic differences (Guth, B. E., et al., 1986, Infect Immun 54:587-589, Guth, B. E., et al., 1986, Infect Immun 54:529-536). Murine experiments demonstrated that certain immunomodulatory activities of LT-IIa and LT-IIb are equivalent or greater than those of CT (Connell, T. D., et al., 1998, Immunology Letters 62:117-120, Martin, M., et al., 2000, Infect. Immun. 68:281-287).

The E. coli heat-labile enterotoxins LT-I, LT-IIa, LT-IIb and CT belong to the AB₅ superfamily of bacterial enterotoxins. Members of this superfamily are related in structure and function (Guth, B. E., et al., 1986, Infect Immun 54:587-589, Guth, B. E., et al., 1986, Infect Immun 54:529-536, Spangler, B. D., 1992, Microbiol. Rev. 56:622-47, van den Akker, F., et al., 1996, Structure 4:665-678). Each of these enterotoxins is an oligomeric protein composed of an A polypeptide which is noncovalently coupled to a pentameric array of B polypeptides. The A polypeptide is enzymatically active and upregulates adenylyl cyclase by catalyzing the ADP-ribosylation of the Gₛα regulatory protein. This modification of Gₛα promotes accumulation of intracellular cAMP which indirectly induces the intoxicated cell to secrete chloride ions and likely modulates other processes for which cAMP is a signaling molecule (Cassel, D., et al., 1977, Proc. Natl. Acad. Sci. U S A 74:3307-3311, Holmes, R. K., et al., 1995,. Bacterial Toxins and Virulance Factors in Disease, vol. 8. Marcel Dekker, Inc., New York, Moss, J., et al., 1979, J. Biol. Chem. 254:11993-11999, Moss, J., et al., 1979, Annu. Rev. Biochem. 48:581-600, Moss, J., et al., 1977, J. Biol. Chem. 252:2455-2457), and which is believed to cause the dehydrating symptoms associated with infection by cholera and certain strains of E. Coli.

The B pentamer mediates binding of LT-IIa, LT-IIb, CT, and LT-I to gangliosides, a heterogeneous family of glycolipids located on the surface of mammalian cells (Sonnino, S., et al., 1986, Chem. Phys. Lipids 42:3-26). CT and LT-I bind with high affinity to GM1 and with lower affinity to ganglioside GD1b; LT-IIa-binds specifically, in descending order of avidity, to gangliosides GD1b, GM1, GT1b, GQ1b, GD2, GD1a and GM3; LT-IIb-binds most avidly to GD1a, and to GM2 and GM3 with much lower affinities (Fukuta, S., et al., 1988, Infect. Immun. 56:1748-1753).

LT-IIa, LT-IIb, CT, and LT-I are potent mucosal and systemic adjuvants capable of eliciting strong immune responses to themselves and to unrelated co-administered antigens (Connell, T. D., et al., 1998, Immunology Letters 62:117-120, (Elson, C. O., 1989, Curr. Top. Microbiol. Immunol. 146:29-33, Martin, M., et al., 2000, Infect. Immun. 68:281-287, McCluskie, M. J., et al., 2001, Vaccine 19:3759-3768, Plant, A., et al., 2004, Curr. Top. Med. Chem. 4:509-519, Sougioultzis, S., et al., 2002, Vaccine 21:194-201). However, use of these enterotoxins as mucosal adjuvants in human vaccines has been inhibited by the toxic activity mediated by their A subunits. Thus, there is an ongoing need for improved enterotoxin-based compositions that can be safely used as adjuvants.

### SUMMARY OF THE INVENTION

The present invention provides a method for enhancing an immunological response to an antigen. The method comprises administering to an individual a composition comprising an i) antigen, and ii) an isolated LT-IIa-B pentamer or a mutant thereof, or an isolated LT-IIb-B pentamer or a mutant thereof, whereby administration of the B pentamer of ii) acts as an adjuvant to enhance the immunological response to the antigen of i).

In the present invention, it was unexpectedly observed that compositions comprising B pentamers (and not their respective A subunits) can enhance an immunological response to an antigen, and that the immunological response is distinct from the immunological response enhanced by intact LT-II holotoxins to the same antigen. In particular, the B pentamers effectively induce proinflammatory cytokine release, but the holotoxins are ineffective at inducing proinflammatory cytokine release under the same experimental conditions. Further, the LT-II holotoxins, but not the B pentamers, downregulate proinflammatory signals and upregulate cytokines with anti-inflammatory properties, and thus may antagonize the distinct immunomodulatory effects of the B pentamers. Further, intact holotoxins, while also exhibiting IgA and IgG adjuvant activity, induced a substantial increase in cAMP production in vitro. In contrast, while the B pentamers also exhibited adjuvant activity for IgA and IgG, significantly less cAMP was produced by cells treated with the B pentamers alone. Therefore, compositions comprising B pentamers which have been isolated from their A subunits or produced recombinantly may be useful for enhancing an immune response to an antigen without eliciting unwanted side effects associated with the use of intact holotoxins. Further, certain B pentamer mutations result in altered or reduced receptor binding, which may reduce their capacity to participate in retrograde trafficking through the olfactory nerve.

### BRIEF DESCRIPTION OF THE FIGURES

FIG. 1 is a graphic representation of cytokine induction by the LT-II toxins and CT. THP-1 cells were incubated for 16 h in the absence or presence of heat-labile enterotoxins (LT-IIa, LT-IIb, and CT; all at 2 μg/ml) or with E. coli LPS (10 ng/ml; positive control). Culture supernatants were assayed for cytokine content by ELISA. Results are presented as means±standard deviations of triplicate determinations. Values that are statistically significantly different (P<0.05) from those of controls treated only with medium are indicated by an asterisk.

FIGS. 2A and 2B are graphic representations of LT-II toxins and CT regulate cytokine induction in activated cells. THP-1 cells were pretreated for 1 h with medium only or with 2-μg/ml concentrations of LT-IIa, LT-IIb, or CT. The cells were subsequently incubated for an additional 16 h with medium only, E. coli LPS (Ec-LPS), P. gingivalis LPS (Pg-LPS), or FimA. Culture supernatants were assayed for TNF-α (FIG. 2A) or IL-1β (FIG. 2B) responses by ELISA. Results are shown as means +standard deviations of triplicate determinations. Asterisks indicate statistically significant (P<0.05) inhibition of TNF-α (FIG. 2A) or enhancement of IL-1β (FIG. 2B) responses in LPS- or FimA-activated cells by the toxins.

FIGS. 3A and 3B are graphicical representations of demonstrating that AB₅ toxins inhibit, whereas their B pentamers promote, IL-8 induction. THP-1 cells were pretreated for 1 h with medium only or with 2-μg/ml concentrations of LT-IIa, LT-IIb, CT, or their respective B pentamers. The cells were subsequently incubated for an additional 16 h with 1 μg of Ec-LPS/ml (FIG. 3A) or were left without further treatment (FIG. 3B). The insert summarizes the results of an independent experiment in which THP-1 cells were incubated for 16 h with medium only or with B pentamers in the absence or presence of 10 μg of polymyxin B (PMB)/ml. Induction of IL-8 release in culture supernatants was assayed by ELISA, and data shown are means±standard deviations of triplicate determinations. (FIG. 3A) Statistically significant (P<0.05) inhibition or enhancement of LPS-induced IL-8 release is indicated by an asterisk or a black circle, respectively. (FIG. 3B) B-pentamerinduced IL-8 responses that are statistically significantly (P<0.05) higher than those corresponding to their respective holotoxins are indicated by asterisks, while IL-8 responses that are statistically significantly (P<0.05) elevated over medium-only-treated controls are indicated by black circles.

FIG. 4 is a graphical representation of data demonstrating that LT-IIbB is a more potent cytokine inducer than LT-IIaB or CTB. THP-1 cells were incubated for 16 h in the absence or presence of LT-IIaB, LT-IIbB, or CTB (all at 2 μg/ml) or with E. coli LPS (10 ng/ml; positive control). Induction of TNF-α, IL-1β, or IL-6 release in culture supernatants was assayed by ELISA. Results are presented as means±standard deviations of triplicate determinations. Values that are statistically significantly different (P<0.05) from those of mediumonly-treated controls are indicated by an asterisk.

FIG. 5 is a graphical representation of data demonstrating that the LT-II toxins and CT synergize with proinflammatory stimuli in IL-10 induction. THP-1 cells were pretreated for 1 h with medium only or with 2-μg/ml concentrations of LT-IIa, LT-IIb, or CT. The cells were subsequently incubated for an additional 16 h with medium only, Ec-LPS, Pg-LPS, or FimA. Induction of IL-10 release in culture supernatants was assayed by ELISA. Results are presented as means±standard deviations of triplicate determinations. Asterisks indicate statistically significant (P<0.05) enhancement of IL-10 induction compared to treatment with proinflammatory stimuli in the absence of LT-II toxins or CT.

FIG. 6 is a graphical representation of data demonstrating that cytokine induction by the LT-IIb B pentamer is regulated by LT-IIb holotoxin. THP-1 cells were incubated for 16 h with medium only, LT-IIbB alone, LT-IIbB plus LT-IIb, or LT-IIb alone (all at 2 μg/ml). Culture supernatants were assayed for cytokine content by ELISA. Results are presented as means±standard deviations of triplicate determinations. Cytokine responses in cells treated with both LT-IIbB and LT-IIb holotoxin that are statistically significantly different (P<0.05) from those for treatment with LT-IIbB alone are indicated by asterisks.

FIG. 7 is a graphical representation of SDS-PAGE separation of purified holotoxins (CT, LT-IIa, and LT-IIb) and their respective B subunits (CTB, LT-IIaB, and LT-IIbB) on 15% polyacrylamide gel. The protein samples were heated and the holotoxins were dissociated into A and B subunits. Numbers to the left of the electrophoretogram indicate the molecular mass (Mᵣ) of protein standards.

FIGS. 8A through FIG. 8D are graphical representations of the effect of anti-TLR mAbs on cytokine induction by enterotoxin B pentamers. THP-1 cells were pretreated for 30 min with medium only or with 10 μg/ml of mAbs to TLR2, TLR4, or with an equal concentration of IgG2a isotype control (IC). The cells were then stimulated for 16 h with 2 μg/ml of LT-IIaB, LT-IIbB, or CTB. To evaluate the degree of effectiveness of the anti-TLR mAbs used, THP-1 cells were also stimulated with 0.2 μg/ml of established TLR agonists (Pam3Cys; TLR2 agonist and E. coli [Ec]-LPS; TLR4 agonist) (FIG. 8D). Induction of IL-8 (FIG. 8A) IL-1β (FIG. 8B), TNF-α (FIG. 8C and FIG. 8D), or IL-6 (FIG. 8C and FIG. 8D) in culture supernatants was assayed by ELISA. Results are presented as means and standard deviations (SDs) of triplicate determinations from a typical experiment. Statistically significant (P<0.05) inhibition by anti-TLR2 in comparison to no treatment or to isotype control treatment is indicated by asterisks. Statistically significant differences between groups treated with anti-TLR2 or anti-TLR4 are indicated by black circles.

FIG. 9 is a graphical representation of data demonstrating TLR1/TLR2 activation by LT-IIaB and LT-IIbB. HEK 293 cells were co-transfected with plasmids encoding a luciferase reporter gene driven by a NF-κB-dependent promoter, and with vectors encoding human TLRs (TLR1 plus TLR2, or TLR2 plus TLR6) or with an empty vector (CMV). After 24 h, the cells were stimulated for 6 h with the indicated molecules (all holotoxins or B pentamers were used at 2 μg/ml; Pam3Cys at 20 ng/ml). Cellular activation is reported as relative luciferase activity. The data (fold increase of luciferase activity over corresponding no-agonist control) are presented as means and SDs of values from four separate assays, three of which were performed in triplicate and one in duplicate. Asterisks indicate statistically significant (P<0.05) cellular activation in comparison with the corresponding no-agonist control. Black circles indicate statistically significant differences between TLR1/TLR2- and TLR2/TLR6-dependent cellular activation by the same agonist.

FIGS. 10A and 10B are graphical representations of data demonstrating cytokine induction by LT-II B pentamers in TLR-deficient mouse macrophages. Macrophages from wild-type mice or mice deficient in TLR2 or TLR4 were stimulated for 16 h with LT-IIaB or LT-IIbB (both at 2 μg/ml), or with known TLR2 (Pam3Cys) or TLR4 (E. coli [Ec]-LPS) agonists (both at 0.2 μg/ml). Induction of TNF-α (A) or IL-6 (B) in culture supernatants was assayed by ELISA. Results are presented as means and SDs of triplicate determinations from a typical experiment. Statistically significantly differences (P<0.05) in cytokine induction by the same agonist in TLR-deficient cells compared to wild-type controls are indicated by asterisks.

FIG. 11 is a graphical depiction of the adjuvant activities of wild type and mutant LT-IIa and LT-IIb holotoxins and the wild type B pentamers in a mouse mucosal immunization model at day 18 after administration of the indicated LT-II molecules.

FIG. 12A is a graphical depiction of ELISA results for salivary IgA production from mice intranasally immunized on days 0, 14, and 28 with 1 microgram of holotoxin (LT-IIa or LT-IIb) or B pentamer (LT-IIaB or LT-IIbB) in the presence of AgI/II.

FIG. 12B. is a graphical depiction of serum IgG production from the mice immunized as in FIG. 12A.

FIG. 13 is a graphical depiction of cAMP activity induced by holotoxins and B pentamers in RAW264.7 macrophage cells.

FIGS. 14A and 14B are photographical representations of protein separation and Western blotting data. (FIG. 14A) SDS-PAGE of purified non His-tagged LT-IIa, His-tagged LT-IIa, and His-tagged LT-IIa(T34I) (lane 1, 2, and 3, respectively), His-tagged LT-IIb, His-tagged LT-IIb(T13I) and non His-tagged LT-IIb (lane 4, 5, and 6) dissociated into the A subunit (˜28 kDa) and B subunit monomers (˜12.5 and 13.5 kDa for non-His-tagged and His-tagged B subunits, respectively). (FIG. 14B) Western blot of non-His-tagged LT-IIa, His-tagged LT-IIa, and His-tagged LT-IIa(T34I) (lane 1, 2, and 3, respectively), His-tagged LT-IIb, His-tagged LT-IIb(T13I), and non-His-tagged LT-IIb (lane 4, 5, and 6, respectively) probed with rabbit polyclonal antibodies to LT-IIa and LT-IIb, respectively. Molecular masses are noted in kilodaltons.

FIG. 15 is a graphical representation of binding data of LT-IIa, LT-IIa(T34I), LT-IIb, and LT-IIb(T13I) to various gangliosides. Polyvinyl plates were coated with 10 ng with purified ganglioside or a mixture of gangliosides. Enterotoxins were allowed to bind to ganglioside-coated plates followed by probing with rabbit polyclonal antibodies. Plates were developed using alkaline phosphatase-conjugated goat anti-rabbit IgG secondary antibody and nitrophenyl phosphate.

FIGS. 16A and 16B are graphical representations of salivary IgA (FIG. 16A) and vaginal IgA (FIG. 16B) antibody responses to AgI/II from mice after i.n. immunization with AgI/II alone or with LT-IIa, LT-IIa(T34I), LT-IIb, LT-IIb(T13I), or CT as adjuvants. Results are reported as the arithmetic means±standard error of mean obtained from immunized mice (n=6-8 mice per group). *, significant differences at P<0.05 compared to LT-IIa.

FIGS. 17A-17C are graphical representation of antibody production data Serum IgA (FIG. 17A), IgG (FIG. 17B), and IgG subclass (FIG. 17C) antibody responses to AgI/II after i.n. immunization of mice with AgI/II alone or with LT-IIa, LT-IIa(T34I), LT-IIb, LT-IIb(T13I), or CT as adjuvants. Results are reported as the arithmetic means±standard error of mean of immunized mice (n=6-8 mice per group). IgG subclasses were examined from mice at day 28. Arrow indicates the time point at which the third booster immunization with 5 μg AgI/II was administered (day 203). *, and ** indicates significant differences at P<0.05 and P<0.01, respectively, compared to LT-IIa.

FIGS. 18A-18D are graphical representations of data for production of IFN-γ and IL-4 by AgI/II-specific lymphoid cells isolated from cervical lymph nodes (FIGS. 18A and 18C) and spleen (FIGS. 18B and 18D) of BALB/c mice immunized i.n. with AgI/II alone or with LT-IIa, LT-IIa(T34I), LT-IIb, LT-IIb(T13I) or CT at a point 40 days after the third immunization (day 60). Cells were stimulated in vitro for 4 days with 5 μg AgI/II. Results are reported as the arithmetic mean values±standard error of mean (n=3). FIG. 18A: ***, significant difference at P<0.001 compared to LT-IIa. FIG. 18B:, *, significant difference at P<0.05 compared to LT-IIa. FIG. 18 C; ****, significant difference at P<0.0001 compared to LT-IIa; ***, significant difference at P<0.001 compared to LT-IIb.

FIGS. 19A-19J are graphical representations of binding of wt and mutant LT-IIa and LT-IIb to lymphoid cells isolated from cervical lymph nodes of naïve BALB/c mice. Histograms were gated on: CD3 (total T cells), CD4+ (Helper T cell), CD8+ (Cytotoxic T cell), B220 (B cell), or CD11b (macrophage). Dead cells were excluded by PI staining. Light lines, binding patterns of LT-IIa(T34I) and LT-IIb(T13I); bold lines, binding patterns of LT-IIa and LT-IIb. A shift to the left in fluorescent intensity indicates decrease or absence of binding of an enterotoxin to the cells.

FIG. 20 is a graphical representation of iInduction of cAMP production in macrophages after treatment with enterotoxin. cAMP was measured in RAW264.7 cells (5×10⁷) after incubation for 4 hr with LT-IIa, LT-IIa(T34I), LT-IIb, LT-IIb(T13I), or CT. Results are reported as the arithmetic mean values±standard error of mean (n=3). *, significant difference at P<0.05 compared to untreated cells **, significant difference at P<0.01 compared to LT-IIb(T13I); *** significant differences at P<0.001 compared to LT-IIa(T34I). The fold increase of cAMP in the treated cells over the untreated cells is denoted at the top of the respective bars.

### DESCRIPTION OF THE INVENTION

The present invention provides a method for enhancing an immunological response to an antigen. The method comprises administering to an individual an effective amount of a composition comprising an antigen and an isolated wild type B pentamer or an isolated mutant B pentamer of the E. coli heat-labile LT-IIa or LT-IIb holotoxins, whereby administration of the isolated B pentamer elicits an adjuvant effect to enhance the immunological response to the antigen.

As used herein, the term “isolated B pentamer” refers to a B pentamer that is not in association with an A subunit. Therefore, an isolated B pentamer may be a B pentamer that has either been biochemically separated from its A subunit, or a B pentamer that has been produced recombinantly.

Thus, compositions comprising either isolated wild type or isolated mutant B pentamers can be utilized in the method of the invention. When mutant B pentamers are used, they may be mutants that abrogate or substantially reduce binding to ganglioside receptors. Further, data presented herein demonstrates that the wild type B pentamers induce significantly less of at least one deleterious biochemical intermediate known to be associated with the symptoms of enterotoxin intoxication. Moreover, administration of either wild type or mutant B pentamers induces unexpectedly distinct and potentially beneficial immunological effects as compared to administration of the respective intact holotoxins.

In more detail, B pentamers of LT-IIa and LT-IIb, but not their respective holotoxins, are demonstrated herein to effectively induce proinflammatory cytokine release from human cells. In contrast, the intact LT-IIa and LT-IIb holotoxins, but not their respective B pentamers, are demonstrated to downregulate proinflammatory cytokines (TNF-α) or chemokines (IL-8) and upregulate cytokines with anti-inflammatory (IL-10) properties, indicating the B pentamers may be superior to the holotoxins in stimulating an adaptive immune response. Data presented herein also strongly implicates the Toll-Like Receptors in cellular activation by the B pentamers. In contrast, the LT-IIa and LT-IIb holotoxins do not significantly activate TLR-expressing cells. Thus, isolated B pentamers unexpectedly have an immunological effect that is not exerted by intact holotoxins.

It is additionally demonstrated herein that mucosal (nasal) administration of isolated LT-IIa-B pentamers or LT-IIb-B pentamers (as well as their respective intact holotoxins) in a mouse model results in strong adjuvant activity at mucosal surfaces against a co-administered antigen. Significantly, an augmented adjuvant response was also induced at distal mucosa (vaginal secretions) by the B pentamers and the intact holotoxins. Further, both isolated B pentamers and the holotoxins exhibit the capacity to augment strong antigen-specific IgG responses in serum when employed as a mucosal adjuvant. However, while the holotoxins induced a large increase in cAMP production in vitro, much less cAMP production was induced by use of the B pentamers alone. Therefore, administration of compositions comprising isolated LT IIa-B pentamers, LT IIb-B pentamers, or mutants thereof, may have important and heretofore unrecognized advantages over their respective intact holotoxins.

Isolated B pentamers of LT-IIa or LT-IIb, and mutants thereof, can be obtained by standard recombinant molecular biology techniques. In this regard, intact holotoxins can be extracted from E. coli cultures and the B pentamers biochemically separated from the A subunits. Alternatively, suitable DNA cloning and mutagenesis methods, as well as procedures for expressing and purifying recombinant proteins are known. (See, for example, (Sambrook et al., 2001, Molecular cloning: a laboratory manual, 3rd ed. Cold Spring Harbor Laboratory Press, New York, N.Y.).

In general, to obtain wild type B pentamers, E. coli genomic DNA can be obtained from an E. coli culture according to standard methods. The DNA encoding the B pentamers can be amplified from the genomic DNA, such as by the polymerase chain reaction, and the amplification products can be cloned into a suitable vector for expression and purification of the B pentamers. (The B pentamers are believed to spontaneously pentamerize in solution under physiological conditions.) The B pentamers can be subsequently extracted and purified from the culture according to standard techniques.

Similarly, DNA sequences encoding mutant B pentamers can be prepared using standard mutagenesis techniques. For this purpose, genomic E. coli DNA encoding wild type B pentaers can be amplified and isolated described above, and the desired mutations can be engineered into the B pentamer DNA coding sequences according to standard methodologies. The mutant B pentamer encoding DNA sequences can then be cloned into a suitable expression vector, expressed and purified from culture in the same manner as the wild type B pentamers.

In one embodiment, a mutant LT-IIa-B with a Thr to Ile substitution at position 34 (termed “LT-IIa-B(T34I)”) is provided.

In another embodiment, a mutant LT-IIb-B with a Thr to Ile substitution at position 13 (termed “LT-IIb-B(T13I)” is provided.

In additional embodiments, suitable B subunit mutants include, for LT-IIa (Connell et al., Infection and Immunity, 60:63-70, 1992), substitutions of I, P, G, N, L, R for T at the 13^{th} position; substitutions of I, P, D, H, N for T at the 14^{th} position; substitutions of A, G, M, H, L, R, Q for T at the 34^{th} position. For B sununits of LT-IIb (Connell et al., Molecular Microbiology 16:21-31, 1995), substitutions of I, K, N for T at the 13^{th} position; and substitutions of I, N, R, M, K for T at the 14^{th} position.

For use as adjuvants, suitably purified wild type or mutant B pentamers can be combined with standard pharmaceutical carriers. Acceptable pharmaceutical carriers for use with proteins and co-administered antigens are described in Remington's Pharmaceutical Sciences (18th Edition, A. R. Gennaro et al. Eds., Mack Publishing Co., Easton, Pa., 1990).

In one embodiment, the antigen AgI/II, which is known to be poorly antigenic, is obtained from Streptococcus mutans cultures or is prepared using recombinant techniques. However, those skilled in the art will recognize that the method of the invention can be used to enhance the immune response to any antigen. Thus, the method can be used to enhance the immunogenicity of cancer vaccines, viral vaccines, bacterial vaccines or parasitic vaccines.

Further, in addition to being used as a co-mingled adjuvant, the B subunits can be used as carriers of antigens chemically coupled to the B pentamers to increase the immune response to the coupled antigen. This is particularly advantageous for mucosal routes of immunization to enhance the delivery of the antigen to the immune response tissues. Examples of antigens that may be coupled in this way include proteins, segments of proteins, polypeptides, peptides, and carbohydrates. Antigens can be coupled to isolated B subunits using a variety of conventional methods ways. For example, proteins, polypeptides, peptides, or carbohydrates can be chemically conjugated to enterotoxin B subunits by means of various well-known coupling agents and procedures, for example: glutaraldehyde, carbodiimide, bis-diazotized benzidine, maleimidobenzoyl-N-hydroxysuccinimide ester, N-succinimidyl-(3-[2-pyridyl]-dithio)propionate, cyanogen bromide, and periodate oxidation followed by Schiff base formation. Further, antigen peptides or polypeptides can be genetically fused to the N-terminus or C-terminus, or inserted into exposed loops of the B subunits, to obtain chimeric B pentamer/antigen molecules, by standard recombinant genetic DNA and protein expression technology.

Compositions comprising isolated B pentamers for use as adjuvants can be administered by any acceptable route. Suitable routes of administration include mucosal (e.g., intranasal, ocular, gastrointestinal, oral (including by inhalation), rectal and genitourinary tract), oral) and parenteral (e.g., intravascular, intramuscular, and subcutaneous injection). A preferred route of administration is intransal mucosal administration.

Those skilled in the art will recognize that the amount of B pentamers included in a pharmaceutical preparation will depend on a number of factors, such as the route of administration and the size and physical condition of the patient. The relative amounts of B pentamers in the pharmaceutical preparations can be adjusted according to known parameters. Further, the compositions comprising the B pentamers can be used in a single administration or in a series of administrations in a manner that will be apparent to those skilled in the art.

The following examples describe the various embodiments of this invention. These examples are illustrative and are not intended to be restrictive.

### EXAMPLE 1

This Example demonstrates engineering and purification of holotoxins and their B subunits. To engineer a His-tagged version of LT-IIa, a fragment encoding a portion of the A polypeptide and the B polypeptide was PCR amplified from pTDC400 (Connell, et al., 1992, Infect. Immun. 60:63-70) using the synthetic oligonucleotides 5′-GATGGGATCCTTGGTGTGCATGGAGAAA G-3′ (SEQ ID NO:1; BamHI site is underlined) and 5′-AAATAAACTAGTTTAGTGGTGG TGGTGGTGGTGTGACTCTCTATCTA ATTCCAT-3′ (SEQ ID NO:2; BcuI site is underlined; His codons are double underlined) as primers. PCR conditions were the following: denaturation at 95° C. for 45 s, annealing at 44° C for 45 s, and extension at 72° C. for 2 min, 30 cycles. After digestion with SacI and BcuI, the resulting PCR fragment was substituted for the SacI/BcuI fragment of pTDC200ΔS. This plasmid was derived from pTDC200 (Connell, et al., 1992, Infect. Immun. 60:63-70) upon removal of a redundant SacI restriction site by partial digestion with SacI, followed by blunting the digested site with Klenow fragment and religation with T4 DNA ligase. The plasmid encoding the LT-IIa holotoxin with a His-tagged B polypeptide was denoted pHN4.

To construct a recombinant plasmid encoding the His-tagged B polypeptide of LT-IIa, pHN4 was digested with SacI and BcuI. The obtained DNA fragment (encoding the B polypeptide) was inserted into pBluescript KSII+ (Stratagene, La Jolla, Calif.) at the SacI/BcuI sites to produce pHN15.

To engineer a His-tagged version of LT-IIb, a fragment carrying the genes for A and B polypeptides was PCR amplified from pTDC100 (Connell, T. D., et al., 1995, Mol. Microbiol. 16:21-31) using the synthetic oligonucleotides 5′-CGGGATCCATGCTCAGGTGAG-3′ (SEQ ID NO:3; BamHI site is underlined) and 5′-GGAATTCTTAGTGGTGGTGGTGGTGGTGTTCTGCCT CTAACTCGA-3′ (SEQ ID NO:4; EcoRI site is underlined; His codons are double underlined). PCR conditions were the following: denaturation at 95° C. for 45 s, annealing at 44° C for 45 s, and extension for 2 min, 30 cycles. After digestion with BamHI and EcoRI, the PCR fragment was ligated into pBluescript KSII+ at the BamHI/E coRI sites to produce pHN1, encoding LT-IIb holotoxin with a His-tagged B polypeptide.

Recombinant plasmid pHN16.1, encoding only the His-tagged B polypeptide of LT-IIb, was engineered by ligating the B-polypeptide-encoding XhoI/EcoRI fragment from pHN1 into pBluescript KSII+ at the XhoI and EcoRI sites.

To engineer a His-tagged version of the B subunit of CT (CTB), a fragment encoding a portion of the A polypeptide and the B polypeptide was PCR amplified from pSBR-CT^{ΔA1} (Hajishengallis, G., et al., 1995, J. Immunol. 154:4322-4332) using the synthetic oligonucleotides 5′-TAAGAGCTCACTCGAGGCTTGGAGGGAAGAG-3′ (SEQ ID NO:5; SacI site is underlined) and 5′-TAACTAGTGCTGAGCTTAGTGGTGGTGGTGGTGGTGTATTTGCCATA CTAATTGC-3′ (SEQ ID NO:6; BcuI site is underlined; His codons-are double underlined) as primers. PCR conditions were the following: denaturation at 95° C. for 45 s, annealing at 44° C. for 45 s, and extension at 72° C. for 1 min, 30 cycles. After digestion with SacI and BcuI, the PCR fragment (corresponding to the B polypeptide) was inserted into pBluescript KSII+ at the SacI/BcuI sites to produce pHN14. CT was purchased from List Biological Laboratories, Campbell, Calif.

The sequence of the wild type LT-IIa-B polypeptide is as follows:

| | |
|---|---|
| MSSKKIIGAFVLMTGILSGQVYAGVSEHFRNICNQTTADIVAGVQLKKYIADVNTNTR | (SEQ ID NO: 7) |
| GlYVVSNTGGVWYIPGGRDYPDNFLSGEIRKTAMAAILSDTKVNLCAKTSSSPNHIWA | |
| MELDRES | |

The first 23 amino acids represent the leader sequence and the sequence of the mature polypeptide is as follows:

| | |
|---|---|
| GVSEHFRNICNQTTADIVAGVQLKKYIADVNTNTRGIYVVSNTGGVWYIPGGRDYPD | (SEQ ID NO: 8) |
| NFLSGEIRKTAMAAILSDTKVNLCAKTSSSPNHIWAMELDRES. | |

The sequence of the wt LT-IIb-B polypeptide is:

| | |
|---|---|
| MSFKKIIKAFVIMAALVSVQAHAGASQFFKDNCNRTTASLVEGVELTKYISDINNNTD | (SEQ ID NO: 9) |
| GMYVVSSTGGVWRISRAKDYPDNVMTAEMRKIAMAAVLSGMRVNMCASPASSPNVI | |
| WAIELEAE. | |

The first 23 amino acids represent the leader sequence and the sequence of the mature polypeptide is as follows:

| | |
|---|---|
| GASQFFKDNCNRTTASLVEGVELTKYISDINNNTDGMYVVSSTGGVWRISRAKDYPD | (SEQ ID NO: 10) |
| NVMTAEMRKIAMAAVLSGMRVNMCASPASSPNVIWAIELEAE | |

All plasmids for LT-II protein production were introduced into E. coli DH5αF'Kan (Life Technologies, Inc., Gaithersburg, Md.). Expression of recombinant holotoxin and B pentamers was induced by isopropyl-β-D-thiogalactoside, and the proteins were extracted from the periplasmic space by using polymyxin B treatment as previously described (Martin, M., et al., 2000, Infect. Immun. 68:281-287). Periplasmic protein extracts were precipitated by addition of ammonium sulfate to 60% saturation (390 g/liter). The precipitate was collected by centrifugation and was dissolved in phosphate-buffered saline (pH 7.4). The dissolved precipitate was dialyzed overnight in phosphate-buffered saline to remove ammonium sulfate, after which the recombinant proteins were purified by means of affinity chromatography using a His·Bind resin column (Novagen, Madison, Wis.) according to a protocol provided by the manufacturer. The eluted fraction was passed through a 0.45-μm-pore-size syringe filter and was further purified by means of gel filtration chromatography (Sephacryl-100; Pharmacia, Piskataway, N.J.) using an ÄKTA-FPLC (Pharmacia). The peak fractions were then concentrated using Vivaspin concentrators (Viva Science, Hanover, Germany). The purity of the recombinant proteins was confirmed by sodium dodecyl sulfate-polyacrylamide gel electrophoresis. All protein preparations were also analyzed by quantitative Limulus amebocyte lysate (LAL) assays (using kits from BioWhittaker, Walkersville, Md., or from Charles River Endosafe, Charleston, S.C.) to measure incidental endotoxin contamination. All holotoxin and B-pentamer preparations were essentially free of LPS (≧0.0064 ng/μg of protein). This was subsequently verified (see Results) in cytokine induction assays, the results of which were unaffected by the presence of the LPS inhibitor polymyxin B (10 jig/ml). Further evidence against contamination with heat-stable contaminants was obtained upon holotoxin or B-pentamer boiling, which destroyed their biological activity. The addition of His tag had no effect on the cytokine-inducing ability of the enterotoxins, as shown in preliminary experiments comparing non-His-tagged and His-tagged molecules (data not shown), which were thus subsequently used in the Examples herein.

Data presented in the Examples herein were evaluated by analysis of variance and the Dunnett multiple-comparison test using the InStat program (GraphPad Software, San Diego, Calif.). Statistical differences were considered significant at the level of P<0.05. Where appropriate, two-tailed t tests were also performed. Experiments were performed with triplicate samples and were performed twice or more to verify the results.

### EXAMPLE 2

This Example demonstrates the effects on cytokine induction of the LT-II holotoxins. Unlike CT or LT-I, LT-II toxins have not been previously examined for their capacity to induce cytokine release in monocytes/macrophages. This possibility was addressed in experiments using human monocytic THP-1 cells, which display a macrophage-like phenotype upon differentiation with phorbol myristate acetate (Auwerx, J., 1991, Experientia, 47:22-31, 16).

To perform THP-1 cell culture and cytokine induction assays, human monocytic THP-1 cells (ATCC TIB-202) were differentiated with 10 ng of phorbol myristate acetate/ml for 3 days in 96-well polystyrene culture plates at 37° C. in a humidified atmosphere containing 5% CO₂. This cell line has been widely used as a model of human monocytes/macrophages (Auwerx, J., 1991, Experientia, 47:22-31). The culture medium consisted of RPMI 1640 (Life Technologies) supplemented with 10% heat-inactivated fetal bovine serum (Life Technologies), 2 mM L-glutamine, 10 mM HEPES, 100 U of penicillin G/ml, 100 μg of streptomycin/ml, and 0.05 mM 2-mercaptoethanol. Differentiated THP-1 cells (1.5×10⁵/well) were washed three times and were used in cytokine induction assays in the absence or presence of bacterial molecules as further specified herein. To determine the effect of toxins on cellular activation by LPS or other stimuli as indicated, the cells were pretreated for 1 hour with the toxins prior to stimulation. When toxins and LPS were added concomitantly to the cell cultures, either approach yielded similar data as further detailed in these Examples.

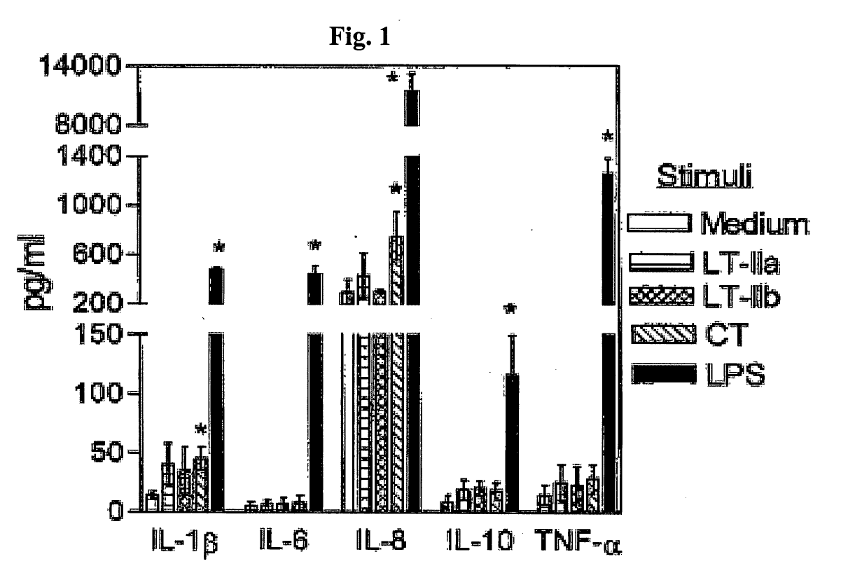

We examined induction of IL-1β, which possesses mucosal adjuvant properties, as well as cytokines that display proin-flammatory (TNF-α), chemotactic (IL-8), immunoenhancing (IL-6), or anti-inflammatory (IL-10) properties. LT-IIa and LT-IIb were tested at 2 μg/ml in comparison with an equal concentration of CT and with 10 ng of Ec-LPS/ml, a potent cytokine-inducing agonist. We found that LT-IIa and LT-IIb did not induce significant release of any of the cytokines tested (FIG. 1). In contrast, CT significantly (P<0.05) yet modestly elevated IL-1 and IL-8 release, whereas Ec-LPS induced high levels of all five cytokines (FIG. 1). LT-IIa and LT-IIb did not induce significant cytokine release even when the dose was increased to 5 μg/ml (data not shown). It should be noted that the enterotoxin preparations were essentially free of LPS.

### EXAMPLE 3

This Example demonstrates the anti-inflammatory activity of the LT-II and CT holotoxins. We investigated whether LT-IIa and LT-IIb actively interfere with the proinflammatory activity of Ec-LPS, a strong TLR4 (Toll Like Receptor-4) agonist. Thus, induction of proinflammatory cytokines by Ec-LPS, a strong Toll-Like Receptor (TLR4) agonist was examined in THP-1 cells pretreated for 1 h with LT-IIa or LT-IIb enterotoxin or with CT. Other proinflammatory virulence factors that activate additional TLRs were also examined to determine whether inhibitory effects by the holotoxins could be extended to those molecules. Specifically, the effect of LPS from P. gingivalis, (Pg-LPS) which activates TLR2, and of recombinant P. gingivalis FimA, which activates TLR2 and TLR4 (Hajishengallis, G., et al., 2004, Infect. Immun. 72:1188-1191, Hajishengallis, G., et al., 2002, Infect. Immun. 70:6658-6664), were also determined.

The fimbrillin subunit (FimA) of Porphyromonas gingivalis fimbriae was purified by means of size-exclusion and anion-exchange chromatography from E. coli BL21 (DE3) transformed with the fimA gene of strain 381 (Hajishengallis, G., et al., 2002, Clin. Diagn. Lab. Immunol. 9:403-411). No LPS activity was detected in the FimA preparation by the LAL assay (BioWhittaker) following chromatography through agarose-immobilized polymyxin B (Detoxi-Gel; Pierce, Rockford, Ill.). LPS was purified from P. gingivalis 381 (Pg-LPS) or E. coli K235 (Ec-LPS) as previously described (Hajishengallis, G., et al., 2002, Infect. Immun. 70:6658-6664), yielding molecules that activate NF-κB exclusively through TLR2 or TLR4, respectively (Hajishengallis, G., et al., 2004, Infect. Immun. 72:1188-1191). The doses used for Ec-LPS, Pg-LPS, and FimA were chosen based on known parameters (Hajishengallis, G., et al., 2004, Infect. Immun. 72:1188-1191, Hajishengallis, G., et al., 2002, Infect. Immun. 70:6658-6664, Hajishengallis, G., et al., 2002, Clin. Diagn. Lab. Immunol. 9:403-411). None of these was found to affect the viability of the cells in the assays, as determined by trypan blue exclusion. Culture supernatants were collected after overnight incubation (16 h) and were stored at −80° C. until assayed. TNF-α, IL-1β, IL-6, IL-8, and IL-10 released into the culture medium were quantitated using enzyme-linked immunosorbent assay (ELISA) kits (purchased from eBioscience, San Diego, Calif., or Cell Sciences, Canton, Mass.) according to protocols recommended by the manufacturers.

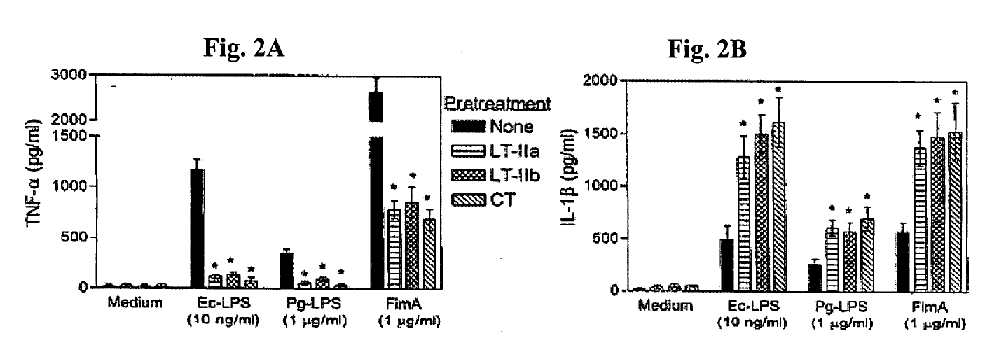

Strikingly, all three holotoxins significantly (P<0.05) inhibited TNF-α induction by all three proinflammatory molecules, especially that by Ec-LPS (≧88% inhibition) (FIG. 2A). In stark contrast, the holotoxins significantly upregulated (P<0.05) IL-1β induction by Ec-LPS, Pg-LPS, or FimA (FIG. 2B). IL-6 induction in activated THP-1 cells was not significantly influenced by any of the holotoxins (data not shown). Cytokine results from this and other experiments described herein were unaffected when the enterotoxins were added to the cells concomitantly with the bacterial stimulants (data not shown) or when the enterotoxins were added to the cells 1 h earlier.

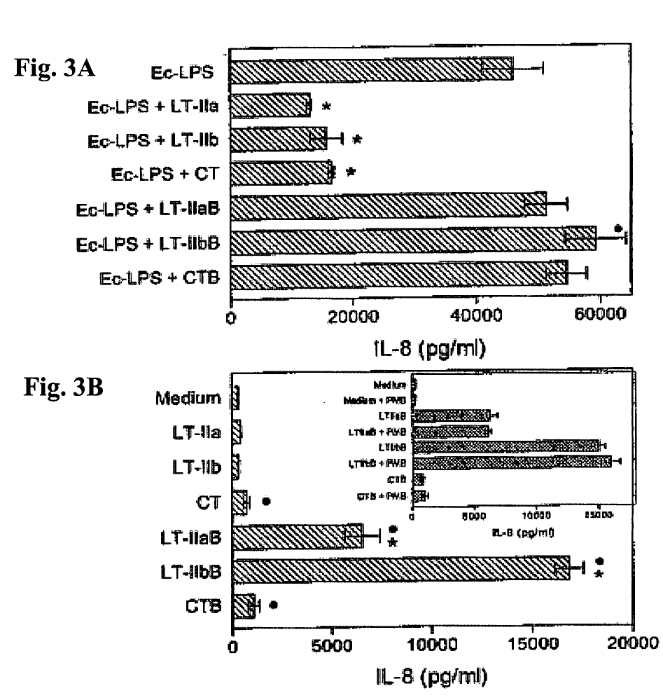

To provide further evidence that the LT-II enterotoxins and CT interfere with inflammatory responses, we examined whether the enterotoxins also inhibit IL-8 induction by Ec-LPS. For this purpose, isolated B pentamers of each enterotoxin were examined in parallel with their respective holotoxins. The LT-II and CT holotoxins significantly (P<0.05) and potently inhibited IL-8 induced in response to a high concentration (1 μg/ml) of Ec-LPS (FIG. 3A), thus confirming their anti-inflammatory potential. In contrast, none of the B pentamers inhibited Ec-LPS-induced IL-8 (FIG. 3A). Instead, the B pentamers appeared to additively augment the Ec-LPS-induced IL-8 response (see also FIG. 3B), although this effect reached statistical significance (P<0.05) for LT-IIb-B only (FIG. 3A). The holotoxins, but not the B pentamers, also inhibited IL-8 induced in response to Pg-LPS (10 μg/ml). The IL-8 response induced by Pg-LPS alone (44,385±2,206 pg/ml) was reduced to 17,894±1,638, 18,004±1,106, or 13,758±611 pg/ml in the presence of LT-IIa, LT-IIb, or CT, respectively. None of the B pentamers could inhibit Ec-LPS-induced TNF-α release (data not shown), in contrast to findings from treatment with the holotoxins (FIG. 2A).

The holotoxins and their B pentamers were also tested alone for their ability to induce IL-8 (FIG. 3B). The holotoxins exhibited either little (CT) or no (LT-IIa and LT-IIb) IL-8-inducing activity, in accordance with results from Example 2 (FIG. 1). Interestingly, however, the isolated B pentamers LT-IIa-B and especially LT-IIb-B induced substantial levels of IL-8 release that were significantly higher (P<0.05) than those induced by their respective holotoxins. Compared to the medium-only control treatment, CTB stimulated a significant (P<0.05) IL-8 release, but this was not significantly higher than the IL-8 response induced by CT (FIG. 3B). Although the purity of the B pentamers with regard to LPS contamination was verified in the LAL assay, to further rule out any stimulatory effects by incidental LPS contamination we repeated the assay of B-pentamer-induced IL-8 in the presence or absence of 10 μg of polymyxin B/ml, the purpose of which is to bind and inhibit the activity of any residual LPS. Polymyxin B had no effect on the ability of any of the B pentamers to stimulate IL-8 production (FIG. 3B insert), whereas it almost completely inhibited IL-8 induction by Ec-LPS (data not shown).

### EXAMPLE 4

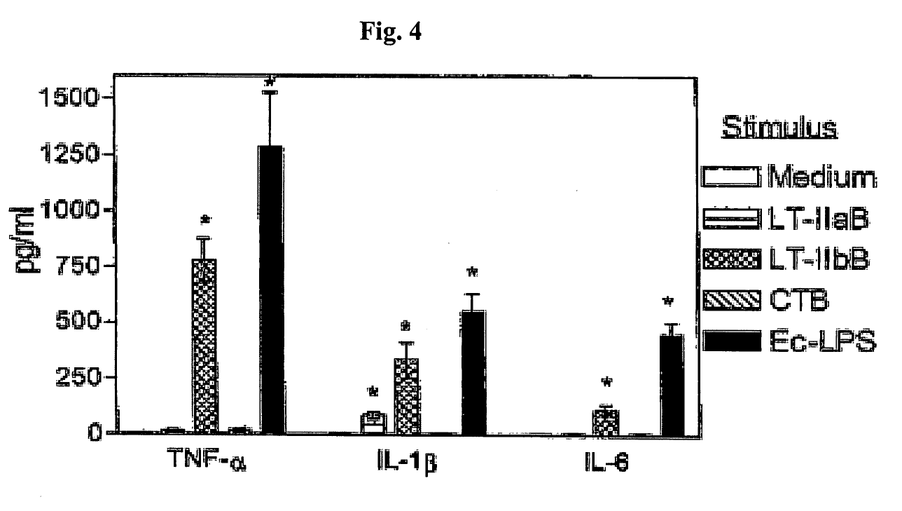

This Example demonstrates particular cytokine induction by the B subunits of LT-IIa and LT-IIb. To determine whether the B pentamers of LT-IIa and LT-IIb induced release of cytokines other than IL-8, THP-1 cells were treated with each B pentamer and the levels of TNF-α, IL-1β, and IL-6 were measured in the culture supernatants. All three cytokines were elicited by treatment with LT-IIb-B. In the case of TNF-α and IL-1β the level of induction was nearly comparable to that induced by application of 10 ng of Ec-LPS/ml (FIG. 4). LT-IIa-B induced a low but detectable amount of IL-1β which was significantly (P<0.05) elevated over that of control cells (FIG. 4). Boiling of the B pentamers for 20 min destroyed their ability to induce cytokines above the levels released by cells treated with medium only (data not shown). This further demonstrated that their effects were not mediated by incidental contamination with LPS in the preparations of purified B pentamers. Treatment of THP-1 cells with CTB did not elicit production of TNF-α, IL-1β, and IL-6 at either 2 βg/ml (FIG. 4) or at 5 βg/ml (data not shown).

Thus, the data presented in FIGS. 1, 3, and 4 collectively indicate that the absence of the A subunit from the LT-II B pentamers facilitates cytokine induction that is distinct from the effects of intact holotoxin.

### EXAMPLE 5

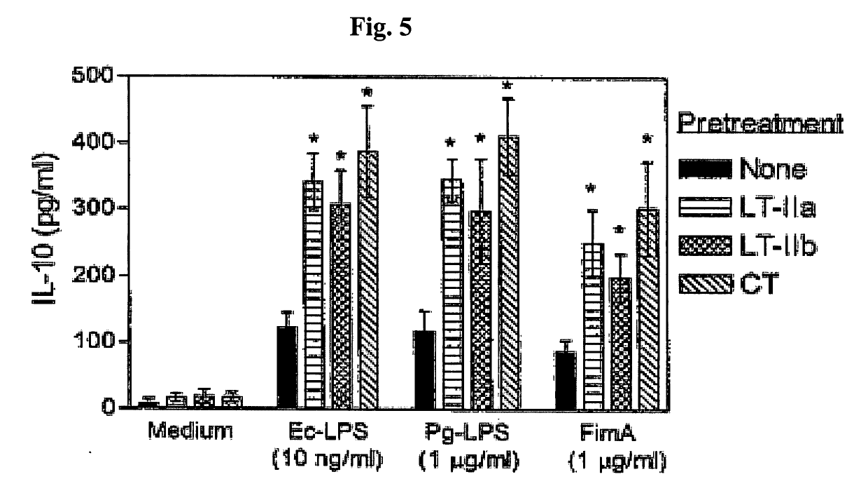

This Example demonstrates the effects of the holotoxins and their respective B subunits on IL-10 induction. We investigated whether LT-II holotoxins and CT inhibition of proinflammatory cytokine induction by Ec-LPS or other bacterial stimuli, such as Pg-LPS and FimA (FIG. 2A and FIG. 3A), may involve IL-10-associated effects. This cytokine is a strong inhibitor of macrophage proinflammatory cytokines (Fiorentino, D., et al., 1991, J. Immunol. 147:3815-3822). Because none of the holotoxins induced significant IL-10 responses in our experimental system (FIG. 1), we determined their ability to augment IL-10 induction by Ec-LPS, Pg-LPS, or FimA. We found that all three toxins significantly (P<0.05) upregulated IL-10 induction by all three bacterial stimuli (FIG. 5). As the enterotoxins had no detectable capacity to induce IL-10 when used alone (FIGS. 1 and 5), it is likely that the observed effects of the enterotoxins in the comixture experiments were synergistic. In contrast, a synergistic effect was not observed when the B pentamers were substituted for the holotoxins in these experiments (data not shown).

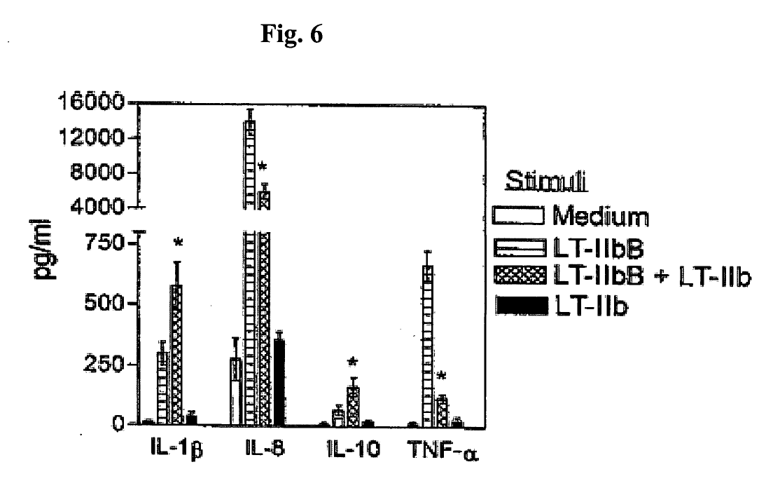

Further analysis of the data indicated that there was a correlation between the ability of the holotoxins to upregulate IL-10 (FIG. 5) and their ability to downregulate TNF-α (FIG. 2A) or IL-8 (FIG. 3A). To confirm this correlation in a single experiment, the effect of LT-IIb holotoxin on LT-IIb-B-pentamer induced IL-10, TNF-α, and IL-8 production (FIG. 6) was determined. Treatment of THP-1 cells with LT-IIb resulted in significant (P<0.05) elevation of IL-10 levels in LT-IIb-B-activated cells which correlated with a decrease in IL-8 and TNF-α levels (FIG. 6). LT-IIb was also found to enhance LTIIb B-pentamer induced IL-1β release (FIG. 6), which was consistent with observations in cells activated with Ec-LPS, Pg-LPS, or FimA (FIG. 2A).

### EXAMPLE 6

This Example provides an analysis of the role of IL-10 in holotoxin-mediated TNF-α and IL-8 downregulation in activated cells. To determine whether the downregulatory effects of the holotoxins on TNF-α and IL-8 induction in activated cells were mediated via induction of IL-10, experiments were conducted using a neutralizing MAb to IL-10 (10 μg/ml) obtained from R&D Systems (Minneapolis, Minn.). If the downregulatory effects were caused by IL-10, then addition of the anti-IL-10 MAb to the cell cultures would be expected to reverse the inhibitory effects of LT-IIa, LT-IIb, or CT on production of these proinflammatory cytokines by cells activated with LT-IIb-B. Although anti-IL-10 significantly (P<0.05) counteracted holotoxin-mediated inhibition of TNF-α or IL-8 induction by LT-IIb-B, the reversal was only partial (Table 1). The use of a higher concentration of anti-IL-10 (20 μg/ml) did not further enhance the reversal effect (data not shown). Similarly, anti-IL-10 only partially reversed holotoxin-mediated inhibition of FimA-induced TNF-α (data not shown). Thus, these data suggest that endogenous production of IL-10 cannot adequately account for the ability of the holotoxins to downregulate proinflammatory cytokine induction. Nonetheless, the data demonstrate that the holotoxins interfere with pro-inflammatory immunological responses.

TABLE 1
Effect of anti-IL-10 on the ability of holotoxins to inhibit
cytokine release in 1T-IIbB activated THP-1 cells^{α}

Amt (pg/ml) of cytokine
released (mean ± SD; n = 3)

| Pretreatment | TNF-α | IL-8 |
|---|---|---|
| None | 908 ± 132 | 12,873 ± 1,347 |
| LT-IIa | 162 ± 47* | 4,912 ± 581* |
| LT-IIa + anti-IL-10 | 286 ± 61** | 6,502 ± 675** |
| LT-IIb | 124 ± 35* | 5,208 ± 740* |
| LT-IIb + anti-IL-10 | 261 ± 67** | 7,009 ± 803** |
| CT | 102 ± 41* | 4,623 ± 419* |
| CT + anti-IL-10 | 232 ± 34** | 6,149 ± 849** |

^{α}THP-1 cells were pretreated for 1 h with holotoxins (either LT-IIa, LT-IIb, or CT; all at 2 μg/ml) in the absence or presence of anti-IL-10 MAb (10 μg/ml). The cells were then stimulated with LT-IIbB (2 μg/ml). After 16 h, culture supernatants were analyzed by ELISA for TNF-α and IL-8 release.
*Statistically significant (P < 0.05) inhibition of LT-IIbB-induced cytokine release by holotoxin.
**Statistically significant (P < 0.05) counteraction of the holotoxin inhibitory effect on LT-IIbB-induced cytokine release. Substitution of isotype-matched control for anti-IL-10 was not statistically different from pretreatment with holotoxin alone (data not shown).

### EXAMPLE 7

This Example demonstrates the effects of LT-II and CT holotoxins on NF-κB activation. Because NF-κB plays a central role in the activation of genes encoding proinflammatory cytokines (Akira, S., 2001, Adv. Immunol., 78:1-56), it was determined whether LT-II enterotoxins and CT downregulate cytokine induction in LT-IIb-B-stimulated cells by interfering with NF-κB activation. Although both p50 and p65 subunits of NF-κB bind target DNA upon NF-κB activation, the p65 subunit was selected for examination because p65 is the transactivating subunit of heterodimeric (p50/p65) NF-κB. THP-1 cells were treated with LT-IIb-B, and the level of activation of NF-κB was measured as described below. FimA was used in a parallel experiment as a positive control for NF-κB p65 activation (Hajishengallis, G., et al., 2004, Infect. Immun. 72:1188-1191), and IL-10 (10 ng/ml) was used as a positive control for inhibition of NF-κB activation (Raychaudhuri, B., et al., 2000, Cytokine 12:1348-1355, Schottelius, A. J. G., et al., 1999, J. Biol. Chem. 274:31868-31874). Briefly, NF-κB activation in THP-1 cells was determined by means of an NF-κB p65 ELISA-based transcription factor assay kit (Active Motif, Carlsbad, Calif.) (Hajishengallis, G., et al., 2004, Infect. Immun. 72:1188-1191, Hajishengallis, G., et al., 2002, Infect. Immun. 70:6658-6664). The detecting antibody used in this ELISA recognizes an epitope on the p65 subunit of NF-κB that is accessible only when NF-κB is activated and bound to its target DNA (containing the NF-κB consensus binding site) attached to 96-well plates. The assay was used to determine LT-IIb-B-induced NF-κB activation and its regulation by holotoxins. Specifically, differentiated THP-1 cells were preincubated at 37° C. for 1 h with culture medium or in the presence of holotoxins as potential downregulators of NF-κB activation. Cells were subsequently stimulated for 90 min with LT-IIb-B. IL-10 was used as a positive control for downregulation of NF-κB activation while FimA was utilized as a positive control for NF-κB activation. Extract preparation and ELISA to detect NF-κB p65 were performed according to the manufacturer's protocols. The optimal time of stimulation and amount of total protein (7.5 μg) used in the ELISA were determined empirically in preliminary experiments.

Results from these experiments indicate that LT-IIb-B did activate NF-κB p65 (Table 2), thus presenting a plausible mechanism for proinflammatory cytokine induction by LT-IIb-B. Boiling of LT-IIb-B at a relatively dilute concentration (<10 μg/ml) to facilitate disassembly of the unusually stable pentameric structure was correlated with a loss in the molecule's ability to activate NF-κB (Table 2).

TABLE 2

| | | Cellular activation assay with: | | |
|---|---|---|---|---|
| | | NF-κB p65 | TNF-α | |
| Stimulus | Pretreatment | (OD₄₅₀) | (pg/ml) | IL-1β (pg/ml) |
| Medium | None | 0.059 ± 0.032 | 9 ± 6 | 7 ± 4 |
| LT-IIb-B | None | 1.132 ± 0.145 | 779 ± 113 | 312 ± 61 |
| | IL-10 | 0.337 ± 0.054* | 103 ± 24* | 71 ± 33* |
| | LT-IIa | 0.848 ± 0.088* | 144 ± 42* | 603 ± 87* |
| | Boiled LT-IIa | 1.278 ± 0.132 | 723 ± 133 | 299 ± 81 |
| | LT-IIb | 0.778 ± 0.074* | 101 ± 65* | 584 ± 103* |
| | Boiled LT-IIb | 1.084 ± 0.077 | 696 ± 157 | 287 ± 88 |
| | CT | 0.812 ± 0.123* | 156 ± 72* | 650 ± 99* |
| | Boiled CT | 1.098 ± 0.101 | 687 ± 183 | 323 ± 45 |
| Boiled LT-IIb-B | None | 0.102 ± 0.047 | 21 ± 10 | 18 ± 9 |
| FimA (positive | None | 1.798 ± 0.286 | 2,474 ± 465 | 343 ± 78 |
| control) | IL-10 | 0.457 ± 0.098* | 482 ± 76* | 92 ± 27* |
| | LT-IIa | 1.352 ± 0.167* | 536 ± 97* | 1,352 ± 282* |
| | Boiled LT-IIa | 1.702 ± 0.208 | 2,547 ± 512 | 387 ± 78 |
| | LT-IIb | 1.211 ± 0.102* | 687 ± 128* | 1,408 ± 335* |
| | Boiled LT-IIb | 1.694 ± 0.187 | 2,163 ± 334 | 362 ± 90 |
| | CT | 1.287 ± 0.129* | 612 ± 110* | 1,208 ± 198* |
| | Boiled CT | 1.762 ± 0.225 | 2,348 ± 292 | 404 ± 98 |

^{α}THP-1 cells were preincubated for 1 h with IL-10 (10 ng/ml) or holotoxins (either LT-IIa, LT-IIb, or CT; all at 2 μg/ml) prior to stimulation with LT-IIbB (2 μg/ml) or FimA (1 μg/ml), which was used as a positive control for NF-κB activation. Boiled LT-IIbB
served as a negative control for stimulus, whereas boiled LT-IIb served as a negative control for pretreatment. After 90 min of stimulation, cellular extracts were analyzed for NF-κB p65 activation by using an ELISA-based kit (Active Motif). After 16 h, culture supernatants
were analyzed by ELISA for TNF-α and IL-1β release. Data shown are means ± standard deviations, n = 3.
*Statistically significant (P < 0.05) differences between non-pretreated controls and groups pretreated with IL-10 or holotoxin. OD₄₅₀, optical density at 450 mm.

This result excludes the possibility that the activation effect was mediated by incidental heat-stable contaminants in the preparation of purified LT-IIb-B. IL-10 significantly (P<0.05) inhibited both LT-IIb-B-mediated activation of NF-κB and the release of TNF-α and IL-1β (Table 2). LT-IIa, LT-IIb, and CT also partially inhibited LT-IIb-B-mediated activation of NF-κB (P<0.05), although the effect was lost when the holotoxins were denatured by boiling (Table 2). It is most likely that the inhibitory effect of the holotoxins on NF-κB activation is IL-10-independent; inhibition of NF-κB p65 activation occurred within 90 min of cellular activation (Table 2), i.e., earlier than release of IL-10 in our experimental system (IL-10 was undetectable after only 2 h of cellular stimulation with LT-IIb-B in the presence or absence of the holotoxins; data not shown). As observed with LT-IIb-B, we found that the holotoxins and IL-10 also regulated FimA-mediated NF-κB activation and cytokine release (Table 2). Thus, this Example demonstrates an intact holotoxin can antagonize the effects of its isolated B pentamer.

### EXAMPLE 8

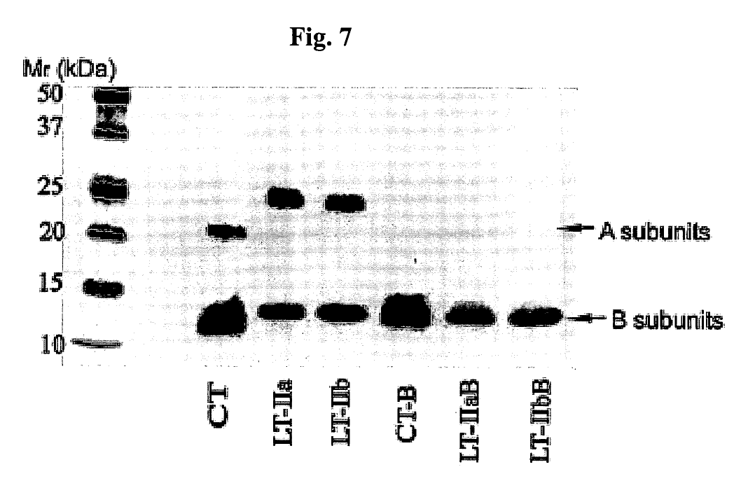

For this and the following Examples, the construction of His-tagged versions of reduced ganglioside binding mutants of LT-IIa-B with a Thr to Ile substitution at position 34 (termed “LT-IIa-B(T34I)”) and of LT-IIb-B with a Thr to Ile substitution at position 13 (termed “LT-IIb-B(T34I)” was performed essentially as described in Example 1, but using pTDC400/T34I (Connell, T., et al., 1992, Infect. Immun. 60:63-70) and pTDC700/T13I, Connell, T. D., et al., 1995, Mol. Microbiol. 16:21-31), respectively, as the starting materials. The resulting plasmids encoding for LT-IIa-B(T34I) and LT-IIb-B(T13I) were denoted pHN22 and pHN19, respectively. The purity of the holotoxins and their respective B subunits was confirmed as specified in Example 1. A representative sodium dodecyl sulfate-polyacrylamide (SDS) gel electrophoresis separation of the purified holotoxins and their B subunits is shown in FIG. 7.

The amino acid sequence of LT-IIb-B(T13I) polypeptide has the sequence shown as SEQ ID NO:11. The complete sequence of LT-IIb and the demonstration that this mutant is non-toxic is available in Connell et al., 1995, Molecular Microbiology, 16:21-31, incorporated herein by reference.

The amino acid sequence of the LT-IIa-B(T34I) mutant is shown as SEQ ID NO:12. The complete sequence of the LT-IIa polypeptide is available as Accession no. M17894 and the complete sequence of the LT-IIb polypeptide is available as Accession no. M28523.

### EXAMPLE 9

This Example demonstrates that TLR2 is involved in B pentamer-induced cytokine release in THP-1 cells. Several microbial proteins appear to display molecular patterns that can activate cells through “Toll-Like Receptors” (TLRs). Whether LT-II B pentamer-induced cellular activation is dependent on TLRs was addressed in cytokine induction assays using THP-1 cells and anti-TLR mAbs. For these experiments, pentameric B subunits of LT-II or CT were used at 2 μg/ml unless otherwise stated. Stimulation was performed in the absence or presence of blocking monoclonal antibodies (mAbs) to TLR2 (TL2.1), TLR4 (HTA125), or immunoglobulin (Ig) isotype-matched (IgG2a) control (e-Bioscience, San Diego, Calif.). None of the molecules was found to affect cell viability as determined by trypan blue exclusion. Culture supernatants were collected after 16-h incubation and stored at −80° C. until assayed for cytokine content using ELISA kits (from eBioscience or Cell Sciences, Canton, Mass.). Similar cell culture procedures were followed to assess cytokine induction (using eBioscience ELISA kits) in mouse peritoneal macrophages from C57BL/6 wild-type mice or mice deficient in TLR2 (Takeuchi, O., et al., 1999, Immunity 11:443-451) or TLR4 (Hoshino, K., et al., 1999, J. Immunol. 162:3749-3752) that have been 9-fold backcrossed on the C57BL/6 genetic background.

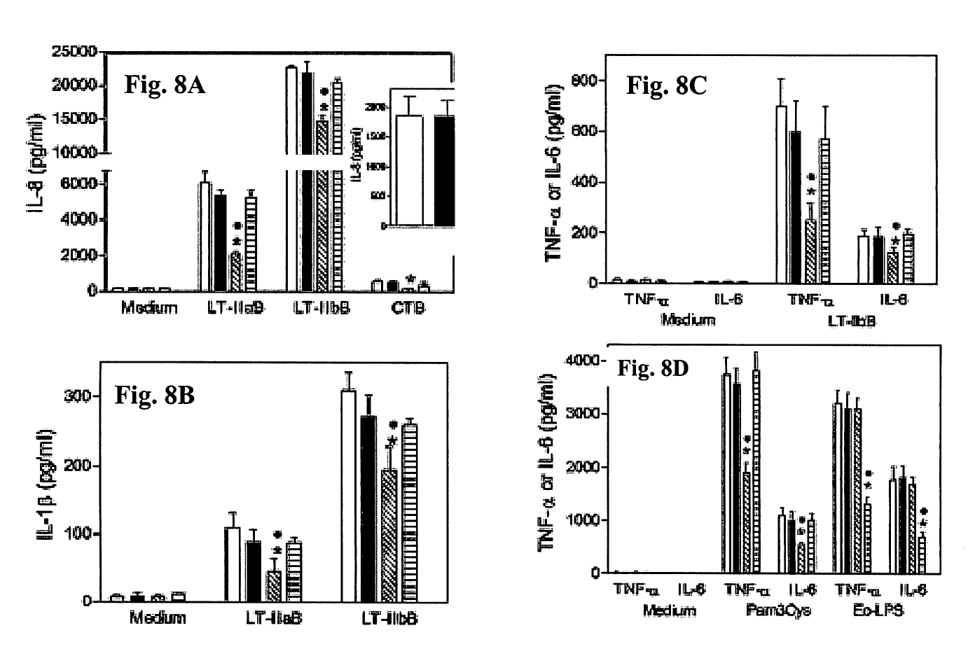

We found that IL-8 induction by LT-IIa-B, LT-IIb-B, or CTB was partially but significantly (P<0.05) inhibited by a mAb to TLR2 (FIG. 8A). CTB was also used at a two-fold higher concentration (4 μg/ml) to enhance induction of IL-8 and thereby to improve evaluation of the inhibitory effect (FIG. 8A insert). Anti-TLR4 mAb or an isotype control had no significant effect on IL-8 induction by the B pentamers (FIG. 8A & insert). Similarly, IL-1β induction by LT-IIa-B or LT-IIb-B was significantly (P<0.05) inhibited by anti-TLR2 but not by anti-TLR4 or isotype control (FIG. 8B; CTB was not tested as it does not induce measurable IL-1β (Hajishengallis, G., et al., 2004, Infect. Immun. 72:6351-6358)). Likewise, anti-TLR2 but not anti-TLR4 inhibited induction of TNF-α and IL-6 release by LT-IIb-B (FIG. 8C); LT-IIa-B and CTB were not tested because they do not induce significant release of these cytokines (Hajishengallis, G., et al., 2004, Infect. Immun. 72:6351-6358). The inhibitory effect of anti-TLR2 mAb was also significant (P<0.05) in comparison to treatment with anti-TLR4 mAb in the case of LT-IIa-B (FIGS. 8A and 8B) or LT-IIb-B (FIG. 8, A to 8C). However, in the case of CTB, the TLR2 mAb effect was not significantly different from that of anti-TLR4 (FIG. 8A and insert). We have thus sought additional, independent approaches to conclusively confirm the role of TLRs in B pentamer-induced cellular activation (see below), as indicated by the TLR mAb data. The degree of effectiveness of the blocking anti-TLR mAbs was monitored in cytokine induction assays using established TLR2 (Pam3Cys) and TLR4 (Ec-LPS) agonists; the obtained results confirmed the specificity of the mAbs although their inhibitory effect was not complete (FIG. 8D).

Induction of IL-8 release in THP-1 cells by 2 μg/ml of LT-IIa-B (4752±611 pg/ml), LT-IIb-B (28530±4367 pg/ml), or CTB (704±84 pg/ml), was unaffected in the presence of 10 μg/ml polymyxin B (corresponding IL-8 responses: 4459±489 pg/ml; 30530±3005 pg/ml; 789±92 pg/ml, respectively) but was abrogated upon boiling of the B pentamers (corresponding IL-8 responses: 147±45 pg/ml; 132±64 pg/ml; 108±28 pg/ml, respectively). Conversely, when THP-1 cells were activated by 0.2 μg/ml of E. coli LPS, the induced IL-8 release (34839±3187 pg/ml) was inhibited by polymyxin B (3098±618 pg/ml) but not by boiling the LPS (37122±5890 pg/ml). These findings verify that activation of the cells by B pentamers was not attributable to contamination with LPS or other heat-stable contaminants.

### EXAMPLE 10

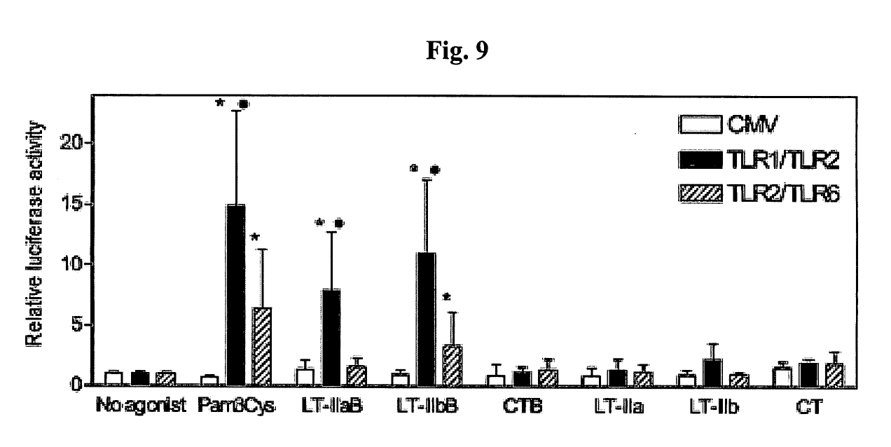

This Example demonstrates that LT-II-B pentamers activate TLR1/TLR2-transfected HEK 293 cells. To further demonstrate TLR2 involvement in B pentamer-induced cellular activation, we used HEK 293 cells transiently cotransfected with cDNAs encoding TLR2 with either TLR1 or TLR6, both of which have been shown to cooperate with TLR2 to mediate signaling (Mielke, P. W., Jr., et al., 1982, Commun. Statist.—Theory Meth. 11: 1427-1437). For these experiments, HEK 293 cells were plated in 24-well tissue culture plates (5×10⁴ cells per well) in 0.5 ml complete RPMI (as above except that 2-mercaptoethanol was not included). The cells were incubated for 16-20 hrs after plating at 37° C. in 5% CO₂ to about 50% confluency. Each well was transfected with 25 ng pRLnull renilla luciferase reporter (Promega, Madison Wis.), 75 ng NF-κB firefly luciferase reporter and one of the following: empty FLAG-CMV vector alone (100 ng), TLR2 (10 ng) and TLR1 (90 ng), or TLR2 (10 ng) and TLR 6 (90 ng). All the TLRs are N-terminal FLAG tagged derivatives of the human receptors. The DNA mixture was mixed with 5 μl CaCl₂ (2.5 M) and sterile water to a volume of 50 μl, after which 50 μl of 2× HEPES-buffered saline was added. The DNA precipitate was then added dropwise to the cells, incubated for 6 hrs at 37° C. in 5% CO₂ after which the media were replaced. Two days after transfection, the cells were stimulated with either no agonist, 20 ng/ml Pam3Cys-Ser-Lys4 lipopeptide (Pam₃Cys; EMC Microcollections, Tuebingen, Germany) or 2 μg/ml of holotoxin or B pentamer preparations. After 16 hrs of stimulation, the media were aspirated and 50 μl of Passive Lysis Buffer (Promega) was added to the plates which were incubated with rocking for 15 minutes at room temperature. Lysates were transferred to a 96-well plate and 10 μl of each lysate was evaluated for luciferase activity using the Dual-Luciferase Reporter Assay System. (Promega). Each firefly luciferase value was divided by the Renilla value to correct for transfection efficiency. All corrected values were normalized to that of no agonist whose value was taken as 1. A non-parametric procedure was used to analyze the data from the luciferase gene reporter assays (FIG. 9) because of significant differences among the standard deviations of the groups under comparison. Specifically, the data from four independent but similar assays were pooled and analyzed by a professional biostatistician using the multi-response permutation procedure for randomized block experiments (MRBP). The analysis was performed using a FORTRAN program (Mielke, P. W., Jr., et al., 1982, Commun. Statist.—Theory Meth. 11:1427-1437). All experimental groups were compared with no-agonist control for TLR1/TLR2 or TLR2/TLR6 activation. The analysis also included comparison of TLR1/TLR2 vs. TLR2/TLR6 activation by the same agonists. Testing was performed at the 0.05 significance level.

Accordingly, HEK 293 cells transfected with TLRs or “empty” control vector were stimulated with LT-IIa-B, LT-IIb-B, CTB, or their respective holotoxins. Pam3Cys, a synthetic TLR2 agonist (Hertz, C. J., et al., 2001, J. Immunol. 166:2444-2450), was used as a positive control. All cotransfections included a cDNA encoding firefly luciferase driven by a NF-κB-dependent promoter in order to monitor cellular activation. We found that, besides Pam3Cys, only LT-IIa-B and LT-IIb-B induced significant (P<0.05) cellular activation upon transfection with TLRs (FIG. 9). LT-IIa-B activated only TLR1/TLR2-transfected cells (FIG. 9). LT-IIb-B additionally activated TLR2/TLR6-transfected cells, although it displayed a significantly higher (P<0.05) capacity to activate cells cotransfected with TLR1 plus TLR2 (FIG. 9). The ability of LT-IIa-B or LT-IIb-B to activate HEK 293 cells was diminished when these were transfected with TLR2 alone (not shown). None of the holotoxins induced significant TLR-dependent activation in HEK 293 cells (FIG. 9), in line with their weak cytokine-inducing capacity observed in earlier experiments using THP-1 cells (Hajishengallis, G., et al., 2004, Infect. Immun. 72:6351-6358). As expected, a TLR4 agonist (E. coli LPS) did not activate either TLR1/TLR2- or TLR2/TLR6-transfected cells (not shown). These results demonstrate a TLR2 requirement in cellular activation by LT-IIa-B or LT-IIb-B and indicate that TLR1 may be a signaling partner of TLR2 in this regard. Thus, this is believed to be the first demonstration that enterotoxin B pentamers cause cellular activation in a TLR-dependent fashion.

### EXAMPLE 11

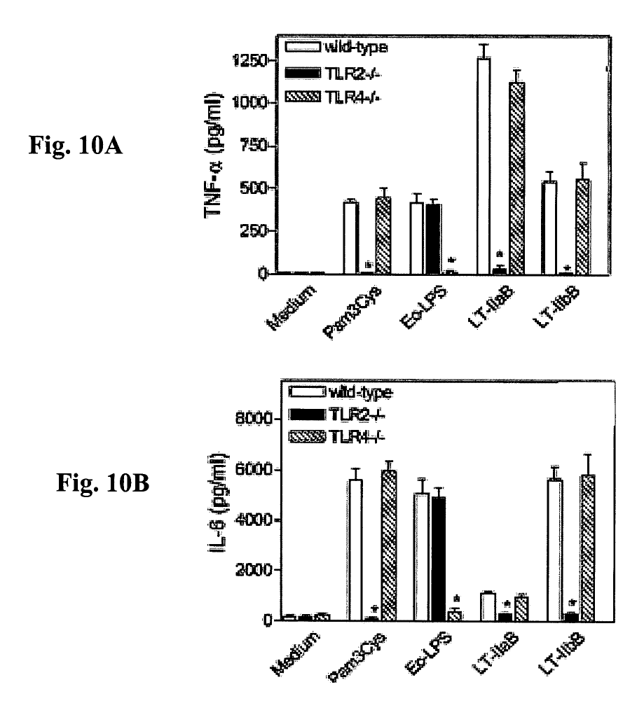

This Example demonstrates that TLR2 is likely required for LT-II B pentamer-induced cytokine release in mouse macrophages. We evaluated the ability of LT-IIa-B or LT-IIb-B to induce cytokine release in TLR2-deficient macrophages compared with wild-type or TLR4-deficient cells. To elicit peritoneal macrophages, mice were injected with 3 to 4 ml of sterile 3% thioglycollate and cells were harvested after 5 days by flushing the peritoneal cavity with 10 ml of ice-cold PBS four times. Isolated cells were then subjected to density gradient centrifugation (Histopaque 1.083) to remove dead cells and red blood cell contamination. Cells were then washed three times with PBS and re-suspended in complete RPMI medium at 1×10⁶/ml. Known TLR agonists (Pam3Cys, TLR2; E. coli LPS, TLR4) were used as positive or negative controls. All control TLR agonists and LT-II B pentamers induced release of TNF-α (FIG. 10A) or IL-6 (FIG. 10B) in wild-type macrophages. Similar to Pam3Cys, however, neither LT-IIa-B nor LT-IIb-B could stimulate substantial cytokine release in TLR2-deficient macrophages, although they were unaffected by TLR4 deficiency (FIG. 10). As expected, the reverse was true for E. coli LPS, which maintained its cytokine-inducing ability in TLR2-deficient but not in TLR4-deficient macrophages (FIG. 4). These results demonstrate that TLR2 is required for LTIIa-B or LTIIb-B-induced activation of mouse macrophages and reinforce similar findings obtained using human cell lines (FIGS. 8 and 9).

### EXAMPLE 12

This Example demonstrates that LT-II B pentamers likely require different ganglioside binding for cellular activation.

Since TLRs often require co-operation with other pattern-recognition [receptors (PRRs) to mediate cellular activation, we determined whether ganglioside binding may be important for the ability of LT-IIa-B or LT-IIb-B to induce TLR2-dependent activation of THP-1 cells. For this purpose we used two mutants, LT-IIa-B(T34I) and LT-IIb-B(T13I), which show no detectable binding to any gangliosides as tested herein, such as GD1a, GD1b, GT1b, GQ1b, GM1, GM2, or GM3 (Connell, T., et al., 1992, Infect. Immun. 60:63-70, (Connell, T. D., et al., 1995, Mol. Microbiol. 16:21-31).

Surprisingly, we found that LT-IIa-B(T34I)was even more effective than the wild-type molecule in inducing cytokine release or NF-κB p65 activation (Table 3; NF-κB activation experiments performed as described in Example 7). Therefore, whereas TLR2 appears to be important for cellular activation by LT-IIa-B (Table 3), gangliosides (at least the ones mentioned above that include those which may be important for LT-IIa toxicity) do not play a role in this regard. On the other hand, the LT-IIb-B(T13I) mutant did not retain any of the proinflammatory activity (cytokine induction or NF-κB p65 activation; Table 3) of the wild-type molecule. Therefore the high-affinity receptor of LT-IIb-B, GD1a, may also be required also for the ability of this molecule to activate THP-1 cells in a TLR2-dependent mode (Table 3).

TABLE 3

| | Receptor | Amt (pg/ml) of cytokine released (means ± SD; n = 3) | | | | NF-κB activation (OD₄₅₀) |
|---|---|---|---|---|---|---|
| Treatment | interference | IL-1β | IL-6 | IL-8 | TNF-α | (means ± SD; n = 3) |
| Medium only | Not applicable | 8 ± 2 | <3 | 32 ± 11 | <6 | 0.098 ± 0.048 |
| LT-IIaB | None | 101 ± 18 | 18 ± 11 | 3,452 ± 323 | <6 | 0.906 ± 0.153 |
| LT-IIaB/T341 | GD1b, GD1a, GM1 | 278 ± 44^{▴} | 36 ± 5 | 9,402 ± 760^{▴} | 39 ± 3^{▴} | 1.348 ± 0.221^{▴} |
| LT-IIaB + anti-TLR2 | TLR2 | 37 ± 5* | 10 ± 6 | 1,391 ± 242* | <6 | 0.401 ± 0.112* |
| LT-IIbB | None | 457 ± 56 | 105 ± 16 | 32,408 ± 922 | 867 ± 81 | 1.642 ± 0.302 |
| LTIIbB/T131 | GD1a | 10 ± 3* | <3* | 211 ± 18* | <6* | 0.134 ± 0.077* |
| LTIIbB + anti-TLR2 | TLR2 | 275 ± 39* | 70 ± 11* | 14,425 ± 1,590* | 282 ± 58* | 0.807 ± 0.176* |
| *THP-1 cells were pretreated for 30 min with anti-TLR2 MAb (10 μg/ml) or medium only prior to stimulation with LT-II B pentamers or nonbinding mutants thereof (all at 2 μg/ml). Induction of cytokine release in culture supernatants, collected 16 h after stimulation, was evaluated by ELISA. In a similar experiment, cellular extracts were prepared | | | | | | |
| # after 90-min stimulation and analyzed for NF-κB p65 activation using an ELISA-based kit (Active Motif). Statistically significant (P < 0.05) enhancement (^{▴}) or inhibition (*) of LT-II B-pentamer-induced cytokine release or NF-κB p65 activation. OD₄₅₀ optical density at 450 nm. | | | | | | |

### EXAMPLE 13

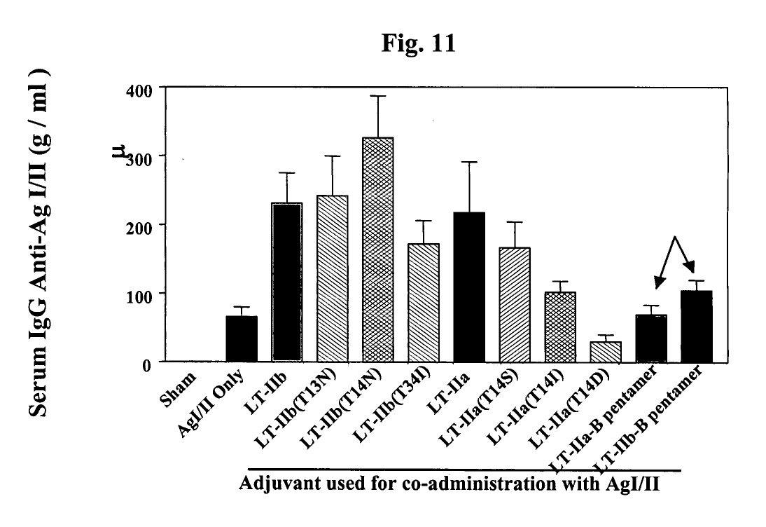

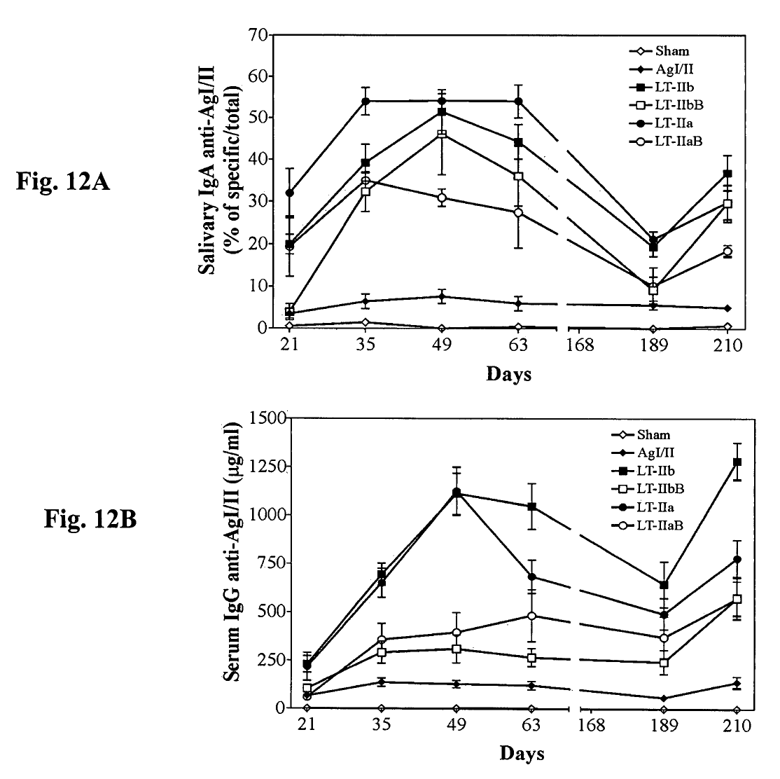

This Example demonstrates the adjuvant activities of wild type and mutant LT-IIa and LT-IIb holotoxins and their respective wild type B pentamers in a mouse mucosal inmmunization model. Mice were intranasally administered LT-II holotoxins or isolated B pentamers as indicated in FIG. 11 in combination with AgI/II, or as indicated for the controls. Sera from the mice were assayed for AgI/I specific IgG levels by ELISA. The results in FIG. 11 are shown only for serum samples taken on Day 18 which is not predicted to be at the peak of the immune response, based on results from prior immunization experiments (data not shown). The arrows denote the antigen-specific immune responses against the antigen after co-administration with the wild type B pentamers of LT-IIa and LT-IIb. The difference between the immune responses against AgI/II of mice immunized with AgI/II and with mice immunized with AgI/II+LT-IIa-B pentamer was significant (p<0.05); at this early time point, there was not a statistical difference in the antigen-specific responses observed between mice receiving AgI/II and mice receiving AgI/II+LT-IIa B pentamer. However, in further experiments, mice were intranasally immunized on days 0, 14, and 28 with 1 microgram of holotoxin (LT-IIa or LT-IIb) or B pentamer (LT-IIa-B or LT-IIb-B) in the presence of AgI/II (10 micrograms). Control mice were immunized with either AgI/II in the absence of holotoxin or B pentamer or were administered only the carrier buffer (sham), as indicated it FIG. 12. The amount of AgI/II-specific IgA as a percent of total IgA was determined by ELISA in saliva collected from the immunized mice at various timepoints. The results from these experiments are summarized in FIG. 12A, which demonstrate that both B pentamers (as well as the holotoxins) exhibit significant adjuvant activity at the mucosal surface, as evidenced by a significant increase in antigen-specific IgA. Additionally, an augmented IgA anti-AgI/II response was also induced at a distal mucosa (vaginal secretions; data not shown) in the mice administered either B pentamer in combination with AgI/II. Further, as can be seen from the results depicted in FIG. 12B, the amounts of AgI/II-specific IgG present in the sera collected from the mice immunized as above demonstrates that the B pentamers have the capacity to augment strong antigen-specific IgG responses in the serum when employed as a mucosal adjuvant.

### EXAMPLE 14

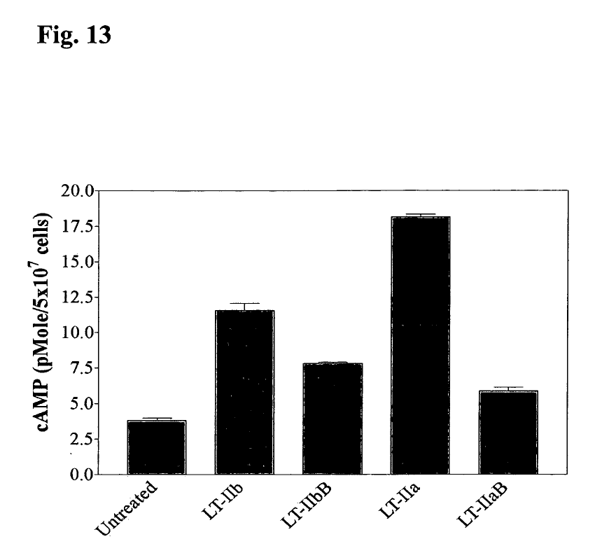

This Example demonstrates the level of cAMP activity induced by holotoxins and B pentamers in RAW264.7 macrophage cells. To conduct these experiments, RAW264.7 macrophage cells (5×107) were treated for 6 hrs with 1 microgram of either holotoxin or B pentamer. The amount of cAMP in the treated cells was measured by a competition ELISA (Cayman Chemicals, Ann Arbor, Mich.). As can be seen from the results depicted in FIG. 13, the holotoxins induced a large increase in cAMP production. In contrast, much less cAMP was produced by cells treated with the B pentamers for which the catalytic A polypeptide is absent. Thus, this Example demonstrates that isolated B subunits are likely to exhibit greatly reduced cAMP production when administered as adjuvants.

### EXAMPLE 15

This Example provides an evaluation of ganglioside-binding activity and adjuvant activity for wild type LT-IIa or LT-IIb holotoxins and for their respective single-point substitution mutants (LT-IIa(T34I) and LT-IIb(T13I). Engineering and purification of His-tagged wild type and mutant LT-II holotoxins for this Example were performed essentially as described in Examples 1 and 8 herein, respectively.

Ganglioside-dependent ELISA. Binding of LT-IIa, LT-IIa(T34I), LT-IIb, or LT-IIb(T13I) to their ganglioside receptors were measured as previously described (Connell, T., et al., 1992, Infect. Immun. 60:63-70, Connell, T. D., et al., 1995, Mol. Microbiol. 16:21-31) with some modifications. Briefly, polyvinyl 96-well ELISA plates were coated overnight at 4° C. with 10 ng GT1b, GQ1b, GM2, GM3, GM₁, GD1a, GD1b, GD2, or with a ganglioside mixture (Matreya, State College, PA and Sigma Chemical Company, St. Louis, Mo.), or with 3.0 μg/ml goat anti-LT-IIa or goat anti-LI-IIb antibodies. After washing and blocking of non-specific binding with 10% horse serum, 50 μl of 1.0 μg/ml of LT-IIa, LT-IIa(T34I), LT-IIb, or LT-IIb(T13I) was added to wells and plates were incubated for 3 hours at 37° C. Unbound enterotoxins were washed away and 50 μl of rabbit anti-LT-IIa or LT-IIb (diluted 1:5000 in PBS+10% horse serum) were added to the wells. Plates were incubated for another two hours at 37° C. and washed to remove unbound antibodies. Fifty μl of 1.0 μg/ml of alkaline phosphatase-conjugated goat anti-rabbit IgG secondary antibody were added to each well. Plates were incubated for one hour at 37° C. after which wells were washed and immediately developed with nitrophenyl phosphate (Amresco, Solon, Ohio) diluted in diethanolamine buffer (100 ml diethanolamine, 1 mM MgCl₂, deionized H₂O to 1 liter; pH 9.8). Color reactions were terminated by adding 50 μl 2.0M NaOH to each well. Optical density of the color reaction was measured at 405 nm.

Toxicity bioassay. The toxicity of purified enterotoxins was measured using Y1 adrenal cells (ATCC CCL-79), a cell line which is acutely sensitive to heat-labile enterotoxins. Briefly, mouse Y1 adrenal cells were cultured to 50% confluence in 96 well tissue culture plates in F-12 medium supplemented with 30% horse serum and 10% fetal bovine serum at 37° C. and in an atmosphere of 5% CO₂. One microgram of CT, LT-IIa, LT-IIa(T34I), LT-IIb, or LT-IIb(T13I) per well was added to the Y1 cell cultures and diluted in a 2-fold dilution series across the plate. Plates were incubated at 37° C. in an atmosphere of 5% CO₂ and examined for 48 hrs to monitor rounding of cells which is an indicator of toxicity. One unit of toxicity is defined as the smallest concentration of enterotoxin that induces rounding of 75 to 100% of the cultured mouse Y1 adrenal cells.

Animals and immunizations. Female BALB/c mice, 11 to 12 weeks of age, were immunized by the intranasal (i.n.) route. Groups of 8 mice were immunized three times at 10-day intervals with AgI/II (10 μg) alone or with AgI/II in combination with 1 μg of CT, LT-IIa, LT-IIa(T34I), LT-IIb, or LT-IIb(T13I). Immunizations were administered in a standardized volume of 10 μl, applied slowly to both external nares. At day 203 after initial immunization all groups were re-immunized i.n. with 5 μg of AgI/II alone. All animal experiments were approved by the Institutional Animal Care and Use Committee at the State University of New York at Buffalo.

Collection of secretions and sera. Samples of serum, saliva, and vaginal washes were collected from individual mice 2 days before the initial immunization (day 0) and at 18, 28, 42, 60, and 175 days after the primary immunization. Saliva samples were collected with a micropipetter after stimulation of salivary flow by injecting each mouse intraperitoneally with 5 μg of carbachol (Sigma). Vaginal washes were collected by flushing the vaginal vault three times with 50 μl of sterile PBS. Serum samples were obtained following centrifugation of blood collected from the tail vein by use of a calibrated capillary tube. Mice were sacrificed at day 217 and blood was collected after cardiac puncture using 20-gauge syringe needles. Mucosal secretions and serum samples were stored at −70° C. until assayed for antibody activity.

Antibody analysis. Levels of isotype-specific antibodies in saliva, sera, and vaginal washes were measured by enzyme-linked immunosorbent assay (ELISA). Polystyrene microtiter plates (96-well; Nunc, Roskilde, Denmark) were coated overnight at 4° C. with AgI/II (5 μg/ml), LT-IIa (3 μg/ml), LT-IIb (3 μg/ml), or CT (3 μg/ml). To determine total immunoglobulin (Ig) isotype concentrations, plates were coated with goat anti-mouse Ig isotype-specific antibodies (Southern Biotechnology Associates, Birmingham, Ala.). Serial twofold dilutions of serum or secretion samples were added in duplicate, and plates were incubated overnight at 4° C. Plates were washed with PBS containing 0.1% Tween-20 (PBS-Tw) and incubated at RT with the appropriate alkaline phosphatase-conjugated goat anti-mouse Ig isotype-specific antibodies (Southern Biotechnology). Plates were washed and developed with nitrophenyl phosphate, as described previously. Concentrations of antibodies and total IgA levels were calculated by interpolation of calibration curves generated by using a mouse Ig reference serum (ICN Biomedicals, Aurora, Ohio). Mucosal IgA responses are reported as the percentage of specific antibody IgA in total IgA to compensate for variations arising from salivary flow rate and dilution of secretions. All enterotoxins were able to induce anti-enterotoxin serum IgG. LT-IIa(T34I) induced lower level of serum IgG than its wild type while LT-IIb(T13I) induced equivalent level of serum IgG as its wild type (data not shown).

Isolation of lymphoid cells. Superficial cervical lymph nodes (CLN) were excised as previously described (Martin, M., et al., 2000, Infect. Immun. 68:281-287). CLN and spleens were teased apart with syringe pistons, dispersed through a 70-μm nylon-mesh screen, and passed twice through 26 gauge syringe needles to obtain single-cell suspensions. Cell suspensions were filtered through nylon mesh to remove tissue debris and centrifuged through Ficoll-Hypaque 1083 (Sigma) to remove erythrocytes and dead cells. All preparations were washed twice and suspended in RPMI 1640 supplemented with 10% fetal bovine serum (FBS). Total cell yield and viability were enumerated in a hemacytometer using trypan blue (Sigma) staining.

Cytokine assays. Spleen and CLN lymphoid cells were plated in triplicates at 5×10⁵ cells per well in flat-bottomed, 96-well tissue culture plates (Nunc), and cultured for 4 days in the presence of concanavalin A (2.5 μg/ml), AgI/II (5 μg/ml) or in the absence of stimulus. Supernatants were collected after centrifugation and stored at −70° C. until assayed for the presence of cytokines. The levels of interleukin-4 (IL-4) and gamma interferon (IFN-γ) in culture supernatants were determined by a cytokine-specific ELISA according to the manufacturer's protocol (Pharmingen, San Diego, Calif.). Briefly, 96-well culture plates were coated with monoclonal anti-IL-4 or anti-IFN-γ (2 μg/ml) and incubated overnight at 4° C. Plates were washed with PBS-Tween and blocked to limit nonspecific binding with 10% FBS in PBS for 1 h at RT. After washing the plates, supernatants were serially diluted in 10% FBS in PBS and added to the wells. A standard curve was generated by using serial dilutions of recombinant IL-4 (500 pg/ml) or IFN-γ (2,000 pg/ml). All serial dilutions were incubated at 37° C. for three hrs followed by washing with PBS-Tween. Secondary antibodies consisted of peroxidase-labeled anti-IL-4 or biotinylated anti-IFN-γ. In assays using biotinylated antibodies, a 1:1,000 dilution of horseradish peroxidase-conjugated streptavidin in 10% FBS in PBS was added to the appropriate wells. After incubation at RT for 2 hrs, reactions were developed for 20 min with o-phenylenediamine-H₂O₂ substrate and terminated by addition of 1.0 M H₂SO₄. The color reaction was measured at 490 nm.

Binding of enterotoxins to CLN lymphoid cells. 10⁶ cells obtained from CLN of naïve mice were treated in vitro with 1.0 μg of LT-IIa, LT-IIa(T34I), LT-IIb, or LT-IIb(T13I). After incubation on ice for 10 minutes, cells were washed and subsequently incubated on ice for 10 minutes with a pre-titrated concentration of polyclonal rabbit antibody to LT-IIa or LT-IIb. After washing, cells were treated with phycoerythrin (PE)-conjugated goat anti-rabbit IgG (0.5 μg/ml) and with fluorescein isothiocyanate (FITC)-conjugated monoclonal antibody to CD3, CD4, CD8, B220, or CD11b. After incubation for 10 minutes on ice, cells were washed and then incubated with 1.0 μg/ml of propidium iodide. CD16/CD32 antibodies were used to block Fc receptor following the manufacturer's instructions. Enterotoxin-binding mutants (1.0 μg), isotype-matched fluorochrome-labeled antibodies, and specific anti-enterotoxin rabbit sera were used as controls to set detection limits. Data acquisition and analysis were performed using a FACScalibur flow cytometer (Beckton-Dickinson, Franklin Lakes, N.J.) and the CellQuest software (Beckton-Dickinson).

Detection of Adenosine 3═,5′ cyclic monophosphate (cAMP). cAMP production was measured in mouse macrophage RAW264.7 cells (ATCC TIB-71) as a relevant lymphoid cell type. Briefly, mouse macrophage RAW264.7 cells (5×10⁷ per well ) were cultured in triplicates for 24 hrs in 24-well tissue culture plates at 37° C. and in atmosphere of 5% CO₂ in Dulbecco's Modified Eagle medium supplemented with 10 mM HEPES, 1 mM sodium pyruvate, 0.1 mM non-essential amino acids, and 10% fetal bovine serum. Culture medium was removed and replaced with fresh culture medium with or without 1.0 μg/ml CT, LT-IIa, LT-IIa(T34I), LT-IIb, or LT-IIb(T13I). After incubation at 37° C. for 4 hrs, enterotoxin-treated cells were extracted with 200 μl of 0.1 M HCl for 20 minutes at RT, scraped from the wells, and centrifuged to clear the extracts of cells and cell debris. Levels of cAMP in the extracts were measured twice using a cAMP EIA·kit (Cayman Chemical Co., Ann Arbor, Mich.) according to the manufacture's protocols.

Statistical analysis. Analysis of variance (ANOVA) and the Tukey multiple-comparison test were used for multiple comparisons. Unpaired t tests with Welch correction were performed to analyze differences between two groups. Statistical analyses were performed using InStat (GraphPad, San Diego, Calif.). Statistical differences were considered significant at the P<0.05 level.

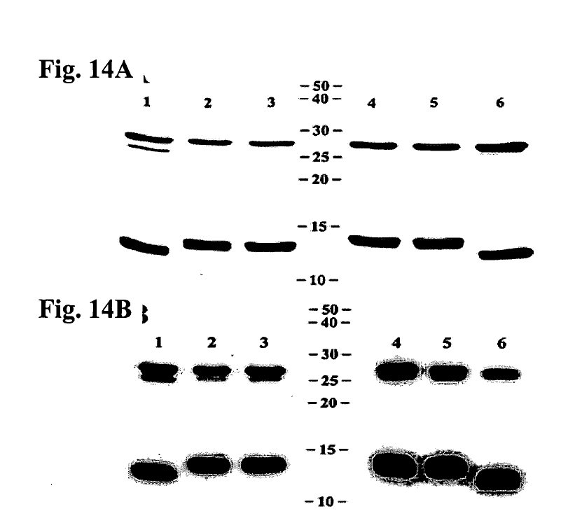

Purification of wt and mutant LT-IIa and LT-IIb. To facilitate their purification, recombinant LT-IIa, LT-IIa(T34I), LT-IIb, and LT-IIb(T13I) holotoxins were engineered with His-tags fused to the carboxyl end of the B pentamers. His-tagged holotoxins were purified from periplasmic extracts of recombinant E. coli using a two-step chromatographic protocol. In the first step, holotoxins and B pentamers were isolated from periplasmic extracts using nickel affinity chromatography. Holotoxins were separated from the contaminating B pentamers by subsequent gel-filtration chromatography. Recombinant wt and mutant holotoxins were examined by sodium dodecyl sulfate-polyacrylamide gel electrophoresis (SDS-PAGE) and immunoblotting using polyclonal antibodies directed toward LT-IIa or LT-IIb to demonstrate that each enterotoxin was purified to apparent homogeneity (FIG. 14). Since experiments to investigate adjuvant properties would be confounded by inadvertent lipopolysaccharide (LPS) contamination of the purified holotoxins, Limulus amoebocyte assays were used to confirm that the purified wt and mutant holotoxins contained less than 0.03 ng of LPS per μg of protein, a level at which LPS-associated immune effects are undetectable in the mouse model (Wu, H. Y., et al., 1998, Vaccine 16:286-292).

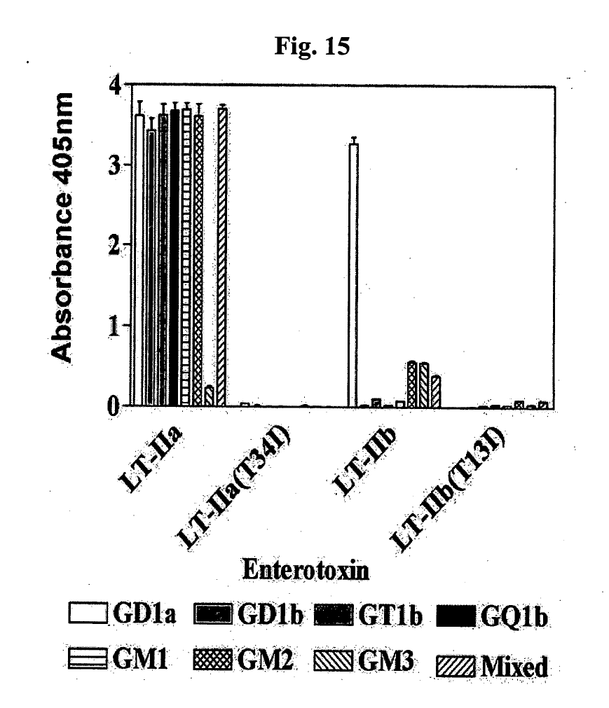

Binding of wt and mutant LT-IIa and LT-IIb to gangliosides. Reduction of binding of LT-IIa(T34I) and LT-IIb(T 13I) to gangliosides was originally defined using periplasmic extracts from recombinant strains of E. coli as crude sources of the mutant enterotoxins (Fukuta, S., et al., 1988, Infect. Immun. 56:1748-1753). To confirm that the ganglioside-binding activities of the purified mutant enterotoxins were equivalent to those of the mutant enterotoxins in the crude extracts, binding of the purified wt and mutant enterotoxins for various gangliosides was measured by ganglioside-specific ELISA (Connell, T., et al., 1992, Infect. Immun. 60:63-70, Connell, T. D., et al., 1995, Mol. Microbiol. 16:21-31) (FIG. 15). LT-IIa bound to gangliosides GD1b, GM1, GT1b, GQ1b, GD2, GD1a and GM3 (Fukuta, S., et al., 1988, Infect. Immun. 56:1748-1753). LT-IIa(T34I), however, exhibited no detectible affinity for those gangliosides (Connell, T., et al., 1992, Infect. Immun. 60:63-70). LT-IIb bound strongly to GD1a and with lower affinity to GM2 and GM3 (Connell, T. D., et al., 1995, Mol. Microbiol. 16:21-31). In contrast, LT-IIb(T13I) had no detectable binding affinity above background for GD1a, GM2, or GM3.

Toxicity of LT-IIa(T34I) and LT-IIb(T13I). Prior results using crude periplasmic extracts from recombinants expressing LT-IIa(T34I) and LT-IIb(T13I) indicated that LT-IIa(T34I) and LT-IIb(T13I) were severely attenuated in toxicity (Connell, T., et al., 1992, Infect. Immun. 60:63-70, Connell, T. D., et al., 1995, Mol. Microbiol. 16:21-31). To confirm those results using purified wild type and mutant holotoxins, Y1 adrenal cell toxicity assays were repeated. Comparisons of the toxicities revealed that CT was the most toxic of the five enterotoxins. Only 0.49 ng of CT was sufficient to induce rounding of 100% of Y1 adrenal cells within a test well. LT-IIa was 16-fold less toxic, requiring 15.65 ng of enterotoxin to cause the same effect. LT-IIa(T34I) exhibited no detectible toxic activity at levels up to 1.0 μg of enterotoxin. Only after 24 hours of incubation with LT-IIa(T34I) was any toxicity detected, i.e. 10% of the cells in the well containing 1.0 μg and 0.5 μg of enterotoxin demonstrating a “rounding” morphology. Y1 adrenal cells had to be incubated with 8-fold the amount of LT-IIb (0.49 ng vs 3.91 ng) to elicit the same degree of toxicity for Y1 adrenal cells as by CT. In comparison, LT-IIb(T13I) was 256-fold less toxic than LT-IIb. In conclusion, the LT-IIa and LT-IIb were significantly less toxic than CT by the Y1 adrenal cell bioassay, and each of the respective mutant enterotoxin was significantly less toxic than its wt parent enterotoxin.

Mucosal adjuvant activities of LT-IIa(T34I) and LT-IIb(T13I). To compare the adjuvant activities of the mutant enterotoxins with the wt enterotoxins, mice were intranasally immunized with AgI/II (Russell, M. W., et al., 1980, Infect. Immun. 28:486-493), in the presence or absence of LT-IIa or LT-IIb. CT was utilized as an external control, as the mucosal adjuvant activities of this enterotoxin for AgI/II have been well-established (Martin, M., et al., 2000, Infect. Immun. 68:281-287, Wu, H. Y., et al., 1998, Vaccine 16:286-292).

Initial immunizations were followed by booster immunizations at day 10 and at day 20. Saliva and vaginal secretions, obtained at intervals up to 175 days after the initial immunization, were analyzed for AgI/II-specific IgA antibodies as a measure of mucosal adjuvant activity of the enterotoxins.

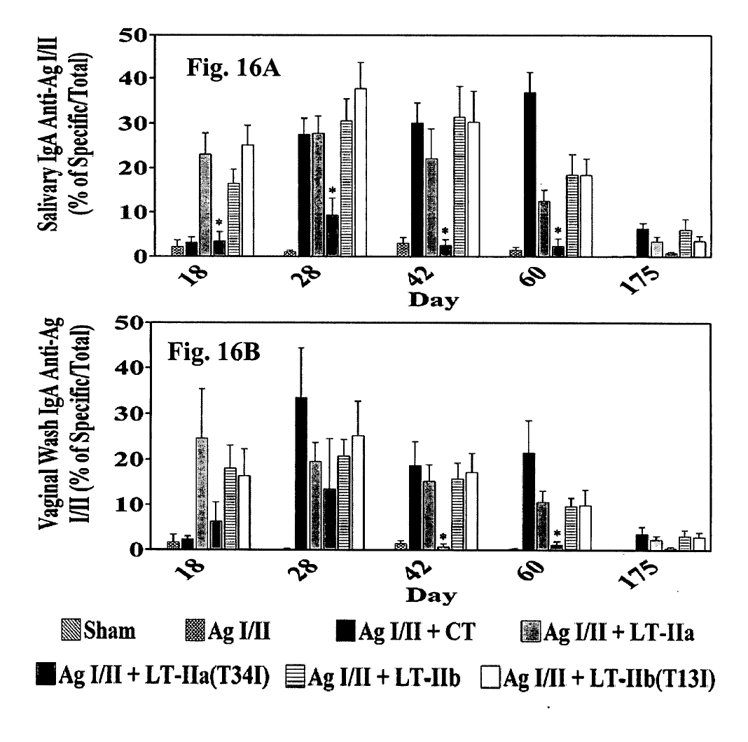

Immunization with AgI/II alone did not elicit a strong salivary IgA response to the antigen (FIG. 16A). In contrast, in mice immunized with AgI/II in the presence of LT-IIa, LT-IIb or CT high levels of AgI/II-specific IgA were detected in the saliva after the second immunization (day 18), peaked at day 28, and persisted, yet declined, through day 175. At all time points, AgI/II-specific salivary IgA levels were 5-fold to 25-fold higher in mice administered AgI/II in the presence of either LT-IIa or LT-IIb. These demonstrate that LT-IIa and LT-IIb were strong mucosal adjuvants (Martin, M., et al., 2000, Infect. Immun. 68:281-287) with capacities for potentiating mucosal anti-AgI/II responses.

When the salivary anti-AgI/II IgA responses of mice immunized with AgI/II+LT-IIa(T34I) were measured, it was found that the mutant enterotoxin was capable of inducing higher mean value of anti-AgI/II IgA antibodies at day 28, but those values were not statistically significant (P>0.05), from mice immunized with AgI/II alone due to high variation among mice (FIG. 16A). Salivary anti-AgI/II IgA responses of those mice were significantly different from the salivary anti-AgI/II IgA of mice immunized with AgI/II+LT-IIa at day 18, 28, 42 and 60 (P<0.05) but not at day 175 (P>0.05). On the other hand, the adjuvant activity was unaffected by the mutation in LT-IIb(T13I) which altered its ganglioside-binding activities. The salivary IgA responses to AgI/II for mice immunized with AgI/II +LT-IIb and for mice immunized with AgI/II+LT-IIb(T13I) were strong and statistically equivalent at all time points (P>0.05)(FIG. 16A).

LT-IIa and LT-IIb when used as intranasal adjuvants were also capable of inducing strong immune responses to a co-administered antigen at distal mucosa (Martin, M., et al., 2000, Infect. Immun. 68:281-287). To determine whether mucosal adjuvant responses were potentiated at distal sites in these experiments, levels of AgI/II-specific IgA was measured in samples taken from the vaginal mucosa (FIG. 16B). Immunization with AgI/II in the absence of enterotoxin did not induce significant amounts of vaginal anti-AgI/II IgA at any time point. In all cases, however, mice administered AgI/II in the presence of LT-IIa, LT-IIb, or CT produced high levels of AgI/II-specific vaginal IgA in comparison to mice receiving only AgI/II (P<0.05)(FIG. 16B) at days 28, 42 and 60. Vaginal IgA responses to AgI/II in those mice receiving an enterotoxin adjuvant peaked at day 28, slowly diminished at later time points, but persisted through day 60 and declined somewhat by day 175. As observed for salivary anti-AgI/II IgA, use of LT-IIa(T34I) as an intranasal mucosal adjuvant induced a higher mean value of vaginal anti-AgI/II IgA than mice immunized solely with Ag I/II, indicating that the mutant enterotoxin retained some mucosal adjuvant activity. In contrast, mice immunized with AgI/II in the presence of LT-IIb(T13I) exhibited a level of vaginal anti-AgI/II which was equivalent to the levels of antigen-specific IgA induced by use of the wt LT-IIb as a mucosal adjuvant (FIG. 16B).

From these results, it was clear that the mucosal adjuvant activity of LT-IIa(T34I) was diminished by reduction of binding affinity for its known ganglioside receptors (e.g. GD1b, GM1, GT1b, GQ1b, GD2, GD1a and GM3). In the case of LT-IIb(T13I), however, the mutation had little or no effect on mucosal adjuvant activity. The mucosal adjuvant activities of LT-IIb(T13I) for inducing antigen-specific IgA, surprisingly, were indistinguishable from the mucosal adjuvant activities of wt LT-IIb.

Systemic adjuvant activity of LT-IIa(T34I) and LT-IIb(T13I). Intranasal administration of LT-IIa, LT-IIb and CT also induces strong circulating antibody responses to co-administered antigens (Connell, T. D., et al., 1998, Immunology Letters 62:117-120, Martin, M., et al., 2000, Infect. Immun. 68:281-287). To examine whether mucosal immunomodulatory activities of LT-IIa(T34I) and LT-IIb(T13I) had the capacity to potentiate serum antibody responses, antigen-specific IgA and antigen-specific IgG were measured in serum samples taken at various time points from mice intranasally immunized with AgI/II in the presence and absence of mutant or wt enterotoxins.

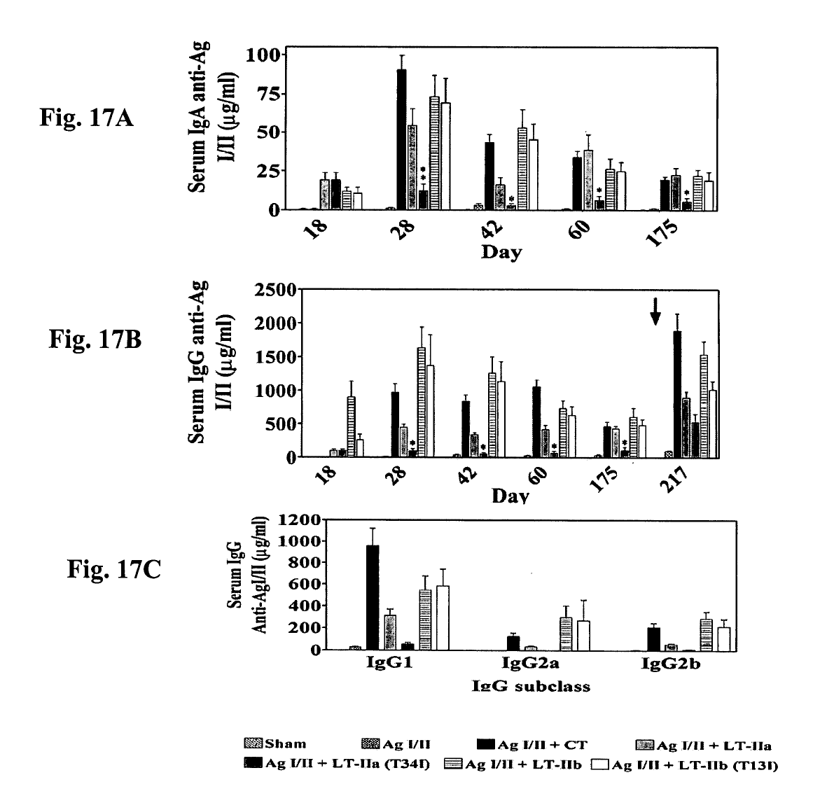

As expected, both LT-IIa and LT-IIb potentiated anti-AgI/II serum IgA after intranasal administration with AgI/II (FIG. 17). As observed for secretory IgA in saliva and vaginal washes, serum IgA (FIG. 17A) responses to AgI/II in mice receiving LT-IIa or LT-IIb as mucosal adjuvants peaked on day 28 and persisted through day 175. In comparison to the serum IgA levels in mice immunized solely with AgI/II, serum IgA responses in mice immunized with AgI/II+LT-IIa (P<0.01), AgI/II+LT-IIb (P<0.001), AgI/II+LT-IIb(T13I) (P<0.001), and AgI/II+CT (P<0.001) were significantly elevated at day 28. Mice receiving LT-IIa(T34I) as a mucosal adjuvant had only a slight elevation in serum IgA level in comparison to mice administered only AgI/II (P<0.05), but this elevation was also significantly diminished from that induced by wt LT-IIa at day 28 (P<0.01) and at days 42, 60 and 175 (P<0.05, respectively). The conclusion from these experiments was that LT-IIa(T34I) was a weak adjuvant for eliciting serum IgA after intranasal application. In contrast, and similar to the patterns observed for salivary and vaginal IgA production, wt LT-IIb and LT-IIb(T13I) had equivalent capacities to induce antigen-specific serum IgA (P>0.05) when used as intranasal adjuvants at all time points.

At all time points tested, serum IgG responses to AgI/II were also elevated in mice immunized with AgI/II+LT-IIa (P<0.05), AgI/II+LT-IIb (P<0.001), and AgI/II+LT-IIb(T13I) (P<0.001) compared to mice immunized with AgI/II alone (FIG. 17B). No significant differences in serum IgG responses were observed between mice immunized with AgI/II and mice immunized with AgI/II+LT-IIa(T34I), although the mean value of the antibody responses was higher in mice immunized with LT-IIa(T34I) as an adjuvant. Boosting with 5 μg AgI/II alone at day 203 i.n. induced 2-fold to 5-fold increases in serum IgG to AgI/II at day 217 in mice administered LT-IIa and LT-IIb compared to the levels of anti-Ag/II IgG at day 175, demonstrating that these enterotoxins stimulated antigen-specific memory responses. When the mice receiving mutant enterotoxins were examined, it was found that there were no significant differences in serum IgG to AgI/II at day 217 between mice immunized with AgI/II+LT-IIb and mice immunized with AgI/II+LT-IIb(T13I). More surprisingly, there was also no statistical difference in AgI/II-specific serum IgG produced in mice immunized with AgI/II+LT-IIa and mice immunized with AgI/II+LT-IIa(T34I). Thus, while LT-IIa(T34I) had only minor ability to potentiate anti-AgI/II immune responses shortly after the initial series of immunizations, this mutant enterotoxin was capable of priming for the recall of antigen-specific immune responses at later time points after boosting.

Serum IgG subclasses responses. Based on IgG subclass distribution, LT-IIb stimulates a more balanced T helper 1 (Th1)/T helper 2 (Th2) immune response than either CT or LT-IIa (Martin, M., et al., 2000, Infect. Immun. 68:281-287). To determine if the mutant enterotoxins stimulated IgG subclass distribution similar or different from those stimulated by their wt parent enterotoxins, the concentrations of AgI/II-specific IgG1, IgG2a, and IgG2b were determined in the serum obtained at day 28. Immunization with AgI/II alone induced low levels of IgG1, IgG2a, IgG2b (FIG. 17C). Levels of IgG subclasses to AgI/II were elevated when AgI/II was co-administered with LT-IIa, LT-IIb, and LT-IIb(T13I), but not when AgI/II was co-administered with LT-IIa(T34I). Consistent with those results, the level of IgG1 was significantly increased in mice immunized with AgI/II+CT in comparison to the levels of IgG2a and IgG2b in mice immunized with AgI/II alone. LT-IIa induced a pattern of AgI/II-specific IgG subclass elevation similar to CT, although the levels were much reduced. IgG1 was the most abundant IgG subclass in mice immunized with AgI/II+LT-IIa, while IgG2a and IgG2b levels were considerably lower. When AgI/II was co-administered with LT-IIb or with LT-IIb(T13I), the levels of IgG1, IgG2a, and IgG2b were significantly increased over that observed in mice immunized solely with AgI/II (FIG. 17C). These data indicate that LT-IIb(T13I) induced a more balanced Th1/Th2 immune response in comparison to either LT-IIa or CT, and similar to the pattern observed when LT-IIb was used as an intranasal adjuvant.

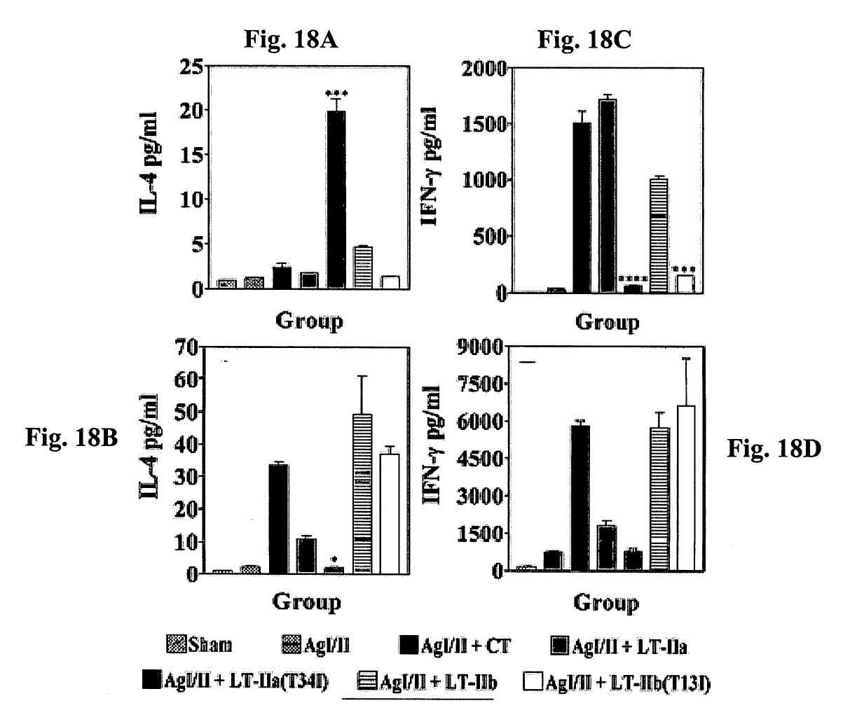

Cytokine production. To complement the IgG subclass distribution experiments, expression patterns for IFN-γ and IL-4 were measured in lymphoid cells obtained from the draining superficial cervical lymph nodes (CLN) and from the spleens of immunized mice after in vitro AgI/II stimulation (FIG. 18). Only low levels of IL-4 were detected in culture supernatants of CLN lymphoid cells of all groups with the exception of culture supernatants of CLN lymphoid cells isolated from mice in which LT-IIa(T34I) was used as an intranasal adjuvant (P<0.001) (FIG. 18A). In contrast, IL-4 was detectable in significantly higher concentrations in culture supernatants of splenic lymphoid cells isolated from mice immunized with AgI/II+LT-IIa (P<0.05), AgI/II+LT-IIb (P<0.001), AgI/II+LT-IIb(T13I) (P<0.01), or with AgI/II+CT (P<0.01) compared to splenic cells from mice immunized with AgI/II without adjuvant or with LT-IIa(T34I) as an adjuvant (FIG. 18B). Very high concentrations of IFN-γ were detected in culture supernatants of CLN lymphoid cells isolated from mice receiving LT-IIa, LT-IIb, or CT as adjuvants compared to mice immunized with AgI/II alone (P<0.0001) (FIG. 18C). IFN-γ concentrations were significantly higher in culture supernatants of CLN lymphoid cells isolated from mice immunized with AgI/II in the presence of LT-IIa (P<0.0001) and LT-IIb (P<0.001) compared to mice immunized with AgI/II in the presence of LT-IIa(T34I) or LT-IIb(T13I), respectively (FIG. 18C). Higher levels of IFN-γ were also detected in culture supernatants of splenic lymphoid cells isolated from mice immunized with AgI/II and CT, LT-IIa, LT-IIa(T34I), LT-IIb, or LT-IIb(I13I) (FIG. 18D). IFN-γ concentrations were significantly higher in culture supernatants of splenic lymphoid cells isolated from mice administered LT-IIa (P<0.05), LT-IIb (P<0.001), LT-IIb(T13I) (P<0.001) or CT (P<0.001) as adjuvants. There was no significant difference between IFN-γ concentrations in culture supernatants of splenic lymphoid cells isolated from mice administered LT-IIa and mice administered LT-IIa(T34I) as adjuvants, or between IFN-γ concentrations in culture supernatants of splenic lymphoid cells isolated from mice immunized with LT-IIb and mice immunized with LT-IIb(T13I) (FIG. 18D).

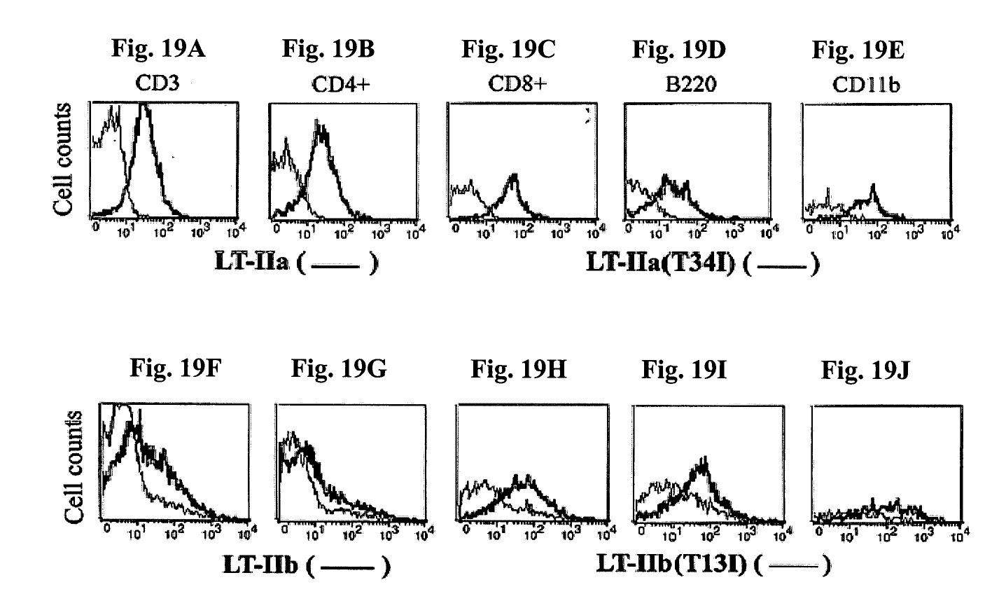

Binding of wt and mutant LT-IIa and LT-IIb to lymphocytes. In vitro binding experiments revealed that LT-IIb(T13I) had little or no detectable binding affinity for ganglioside receptors. Furthermore, exhibits extremely low toxicity for Y1 adrenal cells (Connell, T. D., et al., 1995, Mol. Microbiol. 16:21-31), indicating that the mutant enterotoxin is incapable of inducing production of cAMP, a potent intracellular messenger for a variety of metabolic processes. Thus, we tested whether LT-IIb(T13I) interacts with one or more types of lymphoid cells. To determine whether LT-IIb(T13I) had residual binding affinity for lymphoid cells, cells from the CLN of naïve mice were incubated with wt LT-IIb or with LT-IIb(T13I) and subsequently examined by flow cytometry for bound enterotoxin (FIG. 19). LT-IIb bound to 44.9% of total T cells, 25.3% of CD4+T cells, 83.2% of CD8+T cells, 84.0% of B cells, and 91.5% of macrophages (FIG. 19F-19J). Lesser numbers of all four lymphoid cell types were bound by LT-IIb(T13I), i.e., 13% of total T cells, 8.6% of CD4+T cells, 20.9% of CD8+T cells, 38.4% of B cells, and 44.4% of macrophages (FIG. 19F-19J). In contrast, there was no detectable binding of LT-IIa(T34I) to lymphoid cells (FIG. 19A-19E). The binding of the wild type enterotoxins to different lymphocytes could be inhibited by pre-incubating the enterotoxins with high concentration of their known ganglioside receptors. Pre-incubation of LT-IIb(T13I) had no effect on its ability to bind to lymphocytes (data not shown).

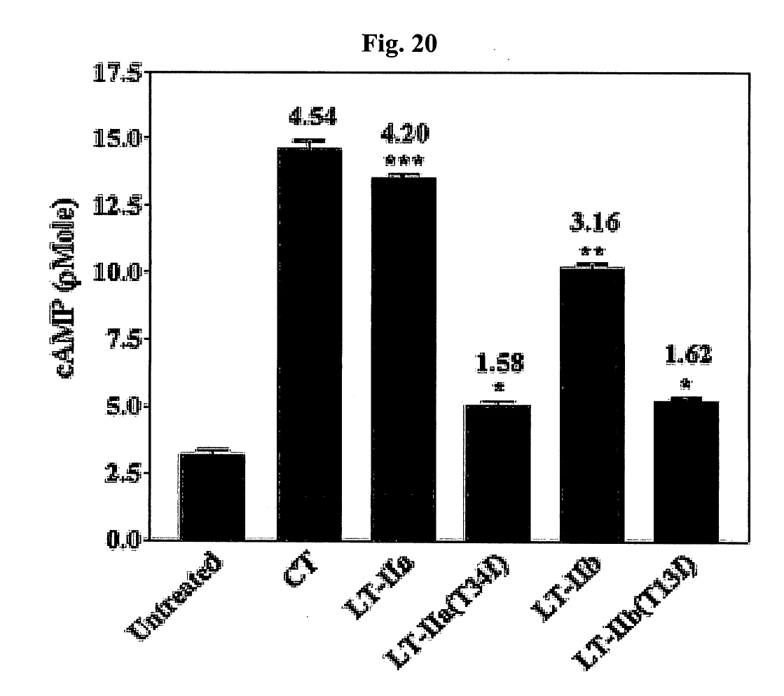

cAMP production in macrophages treated with LT-IIa(T34I) and LT-IIb(T13I). Although LT-IIa(T34I) and LT-IIb(T13I) had no detectable binding in vitro to their major ganglioside receptors (FIG. 15) and exhibited extremely low toxicity for Y1 adrenal cells, our observations that LT-IIb(T13I) bound to lymphoid cells prompted us to determine whether LT-IIb(T13I) and LT-IIa(T34I) retained the capacity to induce cAMP in lymphocytes. Binding assays demonstrated that the LT-IIa(T34I) and LT-IIb(T13I), and their respective wt enterotoxins, bound to RAW 264.7, a mouse macrophage cell line (data not shown) in a similar pattern to CLN macrophages (FIG. 19). To measure cAMP, 5.0×10⁷ cells were incubated for 4 hrs in the presence or absence of each enterotoxin. The endogenous level of cAMP in untreated RAW264.7 cells was 3.22±0.13 pMole. As expected, after incubation with enterotoxins, it was found that LT-IIa, LT-IIb, and CT induced intracellular accumulation of cAMP in RAW 264.7 cells (13.51±0.17, 10.16±0.20 ρMole, and 14.59±0.42, respectively), levels which were 3.2-fold to 4.5-fold higher than observed in untreated cells (FIG. 20). Cells treated with either of the mutant enterotoxins, however, exhibited only slightly elevated amounts of cAMP (1.6-fold) in comparison to the amount of cAMP in untreated cells. The amount of cAMP in cells treated with LT-IIa(T34I) was significantly less than the amount of cAMP induced by treatment of the macrophages with wt LT-IIa (5.20±0.15 ρMole vs 13.51±0.17, (P<0.001). LT-IIb(T13I), which does not have detectable binding in vitro to its known ganglioside receptors using techniques employed herein, and which exhibited little detectable binding to T cells, B cells, or to macrophages from the CLN (FIG. 19), retained a minor capacity to induce production of cAMP in RAW264.7 cells. LT-IIb(T13I) induced significantly less cAMP production than induced by treatment with wt LT-IIa (5.07±0.16 ρMole vs 10.16±0.20 ρMole, P<0.01) (FIG. 20). These data indicated that the capacity of the two mutant enterotoxins to elevate cAMP levels in RAW 264.7 was significantly reduced from the capacity of their respective wt enterotoxins and from CT.

We claim:

1. A method of enhancing an immune response to an antigen in an individual comprising administering to the individual a composition comprising an effective amount of:
a) an isolated LT-IIb-B pentamer or an isolated LT-IIa-B pentamer; and
b) the antigen;
whereby the LT-IIb-B pentamer or the LT-IIa-B pentamer acts as an adjuvant to enhance the immune response to the antigen.

2. The method of claim 1, wherein the LT-IIb-B pentamer is a mutant LT-IIb-B pentamer having a mutation selected from the group consisting of: replacement of threonine by isoleucine, lysine or asparagine at the 13^{th} position; and replacement of threonine by isoleucine, asparagine, arginine, methionine or lysine at the 14^{th} position.

3. The method of claim 2, wherein the mutation of the LT-IIb-B pentamer is a replacement of threonine by isoleucine at the 13^{th} position of the LT-IIb-B pentamer amino acid sequence.

4. The method of claim 1, wherein the LT-IIa-B pentamer is a mutant LT-IIa-B pentamer having mutation selected from the group consisting of: replacement of threonine by isoleucine, proline, glycine, asparagine, leucine or arginine at the 13^{th} position; replacement of threonine by isoleucine, proline, aspartic acid, histidine and asparagine at the 14^{th} position; and replacement of threonine by isoleucine, alkaline, glycine, methionine, histidine, leucine, arginine or glutamine at the 34^{th} position.

5. The method of claim 4, wherein wherein the mutation of the LT-IIa-B pentamer is a replacement of theronine by isoleucine at the 34^{th} position of the LT-IIa-B pentamer amino acid sequence.

6. The method of claim 1, wherein the antigen and the LT-IIb-B pentamer or the LT-IIa-B pentamer are administered mucosally.

7. The method of claim 6, wherein the mucosal administration is selected from the group of routes consisting of intranasal, ocular, gastrointestinal, oral, rectal and genitourinary tract.

8. The method of claim 7, wherein the mucosal administration is intranasal administration.

9. The method of claim 1, wherein the antigen and the LT-IIb-B pentamer or the antigen and the LT-IIa-B pentamer are administered parentally.

10. The method of claim 1, wherein the antigen and the LT-IIb-B pentamer or the the antigen and the LT-IIa-B pentamer are administered via a route selected from the group consisting of intraperitoneal, intravenous, subcutaneous or intramuscular.

11. The method of claim 1, wherein the antigen and the LT-IIb-B pentamer or the antigen and the LT-IIa-B pentamer are administered as a chimeric molecule.

12. The method of claim 1, wherein the antigen and the LT-IIb-B pentamer or the antigen and the LT-IIa-B pentamer are administered as a chemically conjugated molecule.

13. The method of claim 1, wherein the composition further comprises a pharmaceutically acceptable carrier.

14. The method of claim 1, wherein the enhanced immune response is an enhancement of in the production of IgA antibodies, IgG antibodies, or both.

15. The method of claim 14, wherein the IgA antibodies are mucosal IgA antibodies.

16. The method of claim 14, wherein the IgG antibodies are systemic antibodies.


SEQUENCE LISTING (12 sequences)

SEQ ID NO: 1 - DNA, 28 bp, Escherichia coli
  forward LT-IIa-B primer
gatgggatccttggtgtgcatggagaaa

SEQ ID NO: 2 - DNA, 54 bp, Escherichia coli
  reverse LT-IIa-B primer
aaataaactagtttagtggtggtggtggtggtgtgactctctatctaattccat

SEQ ID NO: 3 - DNA, 21 bp, Escherichia coli
  forward LT-IIb-B primer
cgggatccatgctcaggtgag

SEQ ID NO: 4 - DNA, 45 bp, Escherichia coli
  reverse LT-IIb-B primer
ggaattcttagtggtggtggtggtggtgttctgcctctaactcga

SEQ ID NO: 5 - DNA, 31 bp, Vibrio cholerae
  CT-B forward primer
taagagctcactcgaggcttggagggaagag

SEQ ID NO: 6 - DNA, 55 bp, Vibrio cholerae
  CT-B reverse primer
taactagtgctgagcttagtggtggtggtggtggtgtatttgccatactaattgc

SEQ ID NO: 7 - protein, 123 aa, Escherichia coli
  LT-IIa B polypeptide
MSSKKIIGAFVLMTGILSGQVYAGVSEHFRNICNQTTADIVAGVQLKKYIADVNTNTRGIYVVSNTGGVWYIPGGRDYPDNFLSGEIRKTAMAAILSDTKVNLCAKTSSSPNHIWAMELDRES

SEQ ID NO: 8 - protein, 100 aa, Escherichia coli
  LT-IIa B mature polypeptide
GVSEHFRNICNQTTADIVAGVQLKKYIADVNTNTRGIYVVSNTGGVWYIPGGRDYPDNFLSGEIRKTAMAAILSDTKVNLCAKTSSSPNHIWAMELDRES

SEQ ID NO: 9 - protein, 122 aa, Escherichia coli
  LT-IIb B polypeptide
MSFKKIIKAFVIMAALVSVQAHAGASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 10 - protein, 99 aa, Escherichia coli
  LT-IIb B mature polypeptide
GASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 11 - protein, 99 aa, Escherichia coli
  LT-IIb-B(T13I) mutant
GASQFFKDNCNRITASLVEGVELTKYISDINNNTDGMYVVSSTGGVWRISRAKDYPDNVMTAEMRKIAMAAVLSGMRVNMCASPASSPNVIWAIELEAE

SEQ ID NO: 12 - protein, 100 aa, Escherichia coli
  LT-IIa(T34I)mutant
GVSEHFRNICNQTTADIVAGVQLKKYIADVNTNIRGIYVVSNTGGVWYIPGGRDYPDNFLSGEIRKTAMAAILSDTKVNLCAKTSSSPNHIWAMELDRES